(19) United States
(12) Patent Application Publication
(10) Pub. No.: US 2009/0163382 A1
(43) Pub. Date: Jun. 25, 2009

(54) PRIMER SET FOR AMPLIFYING TARGET SEQUENCE(S) OF ANTIBIOTIC-RESISTANT BACTERIAL SPECIES, PROBE OR PROBE SET SPECIFICALLY HYBRIDIZING WITH TARGET SEQUENCE(S) OF ANTIBIOTIC-RESISTANT BACTERIAL SPECIES, METHOD OF DETECTING ANTIBIOTIC-RESISTANT BACTERIAL SPECIES USING THE PROBE OR PROBE SET, AND KIT FOR DETECTING ANTIBIOTIC-RESISTANT BACTERIAL SPECIES

(75) Inventors: Ji-Young OH, Suwon-si (KR); Yeon-su LEE, Goyang-si (KR); Sang-hyun PAEK, Seoul (KR); Byung-chul KIM, Suwon-si (KR); Sook-young KIM, Yongin-si (KR); Kyung-hee PARK, Seoul (KR); Jung-nam LEE, Incheon (KR); Jong-suk CHUNG, Suwon-si (KR); Ah-gi KIM, Yongin-si (KR); Myo-yong LEE, Suwon-si (KR); Tae-jin AHN, Seoul (KR)

Correspondence Address: CANTOR COLBURN, LLP, 20 Church Street, 22nd Floor, Hartford, CT, 06103, US

(73) Assignee: SAMSUNG ELECTRONICS CO., LTD., Suwon-si (KR)

(21) Appl. No.: 11/863,984

(22) Filed: Sep. 28, 2007

(30) Foreign Application Priority Data
Sep. 29, 2006 (KR) 10-2006-0095401
Jan. 24, 2007 (KR) 10-2007-0007628

(51) Int. Cl. C40B 40/08; C07H 21/04

(52) U.S. Cl. 506/17; 536/24.33

(57) ABSTRACT

Provided are a primer set for amplifying target sequence(s) of antibiotic-resistant bacterial species, a probe or probe set specifically hybridizing with target sequence(s) of antibiotic-resistant bacterial species, a microarray immobilized with the probe or probe set, a kit comprising the primer set and a method of detecting at least one antibiotic-resistant bacterial species using the probe or probe set.

### CROSS-REFERENCE TO RELATED PATENT APPLICATION

This application claims priority from Korean Patent Application Nos. 10-2006-0095401, filed on Sep. 29, 2006 and 10-2007-0007628, filed on Jan. 24, 2007 in the Korean Intellectual Property Office, the disclosure of which is incorporated herein in its entirety by reference.

### BACKGROUND OF THE INVENTION

1. Field of the Invention

The present invention relates to a primer set for amplifying target sequence(s) of antibiotic-resistant bacterial species, a probe or probe set specifically hybridizing with target sequence(s) of antibiotic-resistant bacterial species, a microarray immobilized with the probe or probe set, a kit comprising the primer set, and a method of detecting antibiotic-resistant bacterial species using the probe or probe set.

2. Description of the Related Art

Probes for the detection of respiratory disease-associated bacteria are currently known. For example, U.S. Pat. No. 5,830,654 discloses hybridization assay probes for Haemophilus influenzae comprised of an oligonucleotide of about 14-18 nucleotides. U.S. Pat. No. 5,525,718 discloses oligonucleotides selectively hybridizing with a specific gene (e.g., the entE gene) of Staphylococcus aureus. U.S. Pat. No. 6,001,564 discloses primers or probes specific to Escherichia coli, Klebsiella pneumoniae, Pseudomonas aeruginosa, Proteus mirabilis, Streptococcus pneumoniae, Staphylococcus aureus, Staphylococcus epidermis, Haemophilus influenzae, and Moraxella catarrhalis.

In spite of the above-described conventional techniques, no primer sets capable of amplifying target sequences found in antibiotic resistance genes of antibiotic-resistant bacterial species known to be associated with respiratory disease are reported. Furthermore, no probes specific to the target sequences of the antibiotic resistance genes of the antibiotic-resistant bacterial species are reported.

Two single strands of a nucleic acid comprised of nucleotides hybridize to form a double helical structure in which the two polynucleotide chains running in opposite directions are held together by hydrogen bonds between matched base pairs. In a case where a first single strand of a nucleic acid is sufficiently complementary to a second single strand of the nucleic acid, the two single strands are held together under conditions that promote their hybridization, thereby resulting in double-stranded nucleic acid. Under appropriate conditions, DNA/DNA, RNA/DNA, or RNA/RNA hybrids may be formed.

Broadly, there are two fundamental nucleic acid hybridization procedures. In one procedure, known as “in-solution” hybridization, both a “probe” nucleic acid sequence and a nucleic acid molecule of a test sample are free in solution. In the other procedure, a sample nucleic acid is usually immobilized on a solid substrate and a probe sequence is free in solution.

A probe may be a single-stranded nucleic acid sequence which is complementary in some particular degree to a nucleic acid sequence (“target sequence”) sought to be detected. A probe may be labeled. The use of nucleic acid hybridization as a procedure for the detection of particular nucleic acid sequences is disclosed in U.S. Pat. No. 4,851,330, and No. 5,288,611, the disclosures of which are incorporated herein in their entireties by reference.

### SUMMARY OF THE INVENTION

The present invention provides a primer set capable of amplifying target sequence(s) of antibiotic-resistant bacterial species.

The present invention also provides a probe or probe set for detecting at least one antibiotic-resistant bacterial species, which is specific to target sequence(s) amplified using the primer set.

The present invention also provides a microarray immobilized with the probe or probe set and a kit comprising the primer set.

The present invention also provides a method of simultaneously detecting at least one antibiotic-resistant bacterial species using the probe or probe set.

### BRIEF DESCRIPTION OF THE DRAWINGS

The above and other features and advantages of the present invention will become more apparent by describing in detail exemplary embodiments thereof with reference to the attached drawings in which:

FIG. 1 is an image showing the results of PCR products obtained by single PCR and multiplex PCR of five target sequences;

FIGS. 2A, 2B and 2C are images showing the results of PCR products obtained by single PCR and multiplex PCR of 21 target sequences;

FIGS. 3A and 3B are images showing hybridization results of PCR products obtained by PCR using, as primers, a primer set including 21 oligonucleotide sets, and, as templates, genomic DNAs of predetermined antibiotic-resistant bacterial species, on a microarray having a specific oligonucleotide probe layout as presented in Table 7; and

FIG. 3C is an image showing hybridization results of PCR products obtained by PCR using, as primers, a primer set including five oligonucleotide sets, and, as templates, genomic DNAs of antibiotic-resistant bacterial species, on a microarray having a specific oligonucleotide probe layout as presented in Table 8.

### DETAILED DESCRIPTION OF THE INVENTION

The present invention provides an oligonucleotide primer set for amplifying at least one target sequence selected from aataph, ant, aph, CMY1, CMY2, CTX1, CTX2, DHA, IMP, OXA, PER, SHV, TEM, VIM, ermA, ermB, ermC, mef, mecA, Spn pbp2b, Pae gyrA, Sau gyrA, Sau parC, Sau parE, vanA, and vanB genes, the oligonucleotide primer set including at least one oligonucleotide set selected from the group consisting of: an oligonucleotide set including at least one oligonucleotide selected from the group consisting of oligonucleotides which include a fragment of at least 10 contiguous nucleotides present in a nucleotide sequence as set forth in SEQ ID NO: 1 and at least one oligonucleotide selected from the group consisting of oligonucleotides which include a fragment of at least 10 contiguous nucleotides present in a nucleotide sequence as set forth in SEQ ID NO: 2; an oligonucleotide set including at least one oligonucleotide selected from the group consisting of oligonucleotides which include a fragment of at least 10 contiguous nucleotides present in a nucleotide sequence as set forth in SEQ ID NO: 3 and at least one oligonucleotide selected from the group consisting of oligonucleotides which include a fragment of at least 10 contiguous nucleotides present in a nucleotide sequence as set forth in SEQ ID NO: 4; an oligonucleotide set including at least one oligonucleotide selected from the group consisting of oligonucleotides which include a fragment of at least 10 contiguous nucleotides present in a nucleotide sequence as set forth in SEQ ID NO: 5 and at least one oligonucleotide selected from the group consisting of oligonucleotides which include a fragment of at least 10 contiguous nucleotides present in a nucleotide sequence as set forth in SEQ ID NO: 6; an oligonucleotide set including at least one oligonucleotide selected from the group consisting of oligonucleotides which include a fragment of at least 10 contiguous nucleotides present in a nucleotide sequence as set forth in SEQ ID NO: 7 and at least one oligonucleotide selected from the group consisting of oligonucleotides which include a fragment of at least 10 contiguous nucleotides present in a nucleotide sequence as set forth in SEQ ID NO: 8; an oligonucleotide set including at least one oligonucleotide selected from the group consisting of oligonucleotides which include a fragment of at least 10 contiguous nucleotides present in a nucleotide sequence as set forth in SEQ ID NO: 9 and at least one oligonucleotide selected from the group consisting of oligonucleotides which include a fragment of at least 10 contiguous nucleotides present in a nucleotide sequence as set forth in SEQ ID NO: 10; an oligonucleotide set including at least one oligonucleotide selected from the group consisting of oligonucleotides which include a fragment of at least 10 contiguous nucleotides present in a nucleotide sequence as set forth in SEQ ID NO: 11 and at least one oligonucleotide selected from the group consisting of oligonucleotides which include a fragment of at least 10 contiguous nucleotides present in a nucleotide sequence as set forth in SEQ ID NO: 12; an oligonucleotide set including at least one oligonucleotide selected from the group consisting of oligonucleotides which include a fragment of at least 10 contiguous nucleotides present in a nucleotide sequence as set forth in SEQ ID NO: 13 and at least one oligonucleotide selected from the group consisting of oligonucleotides which include a fragment of at least 10 contiguous nucleotides present in a nucleotide sequence as set forth in SEQ ID NO: 14; an oligonucleotide set including at least one oligonucleotide selected from the group consisting of oligonucleotides which include a fragment of at least 10 contiguous nucleotides present in a nucleotide sequence as set forth in SEQ ID NO: 15 and at least one oligonucleotide selected from the group consisting of oligonucleotides which include a fragment of at least 10 contiguous nucleotides present in a nucleotide sequence as set forth in SEQ ID NO: 16; an oligonucleotide set including at least one oligonucleotide selected from the group consisting of oligonucleotides which include a fragment of at least 10 contiguous nucleotides present in a nucleotide sequence as set forth in SEQ ID NO: 17 and at least one oligonucleotide selected from the group consisting of oligonucleotides which include a fragment of at least 10 contiguous nucleotides present in a nucleotide sequence as set forth in SEQ ID NO: 18; an oligonucleotide set including at least one oligonucleotide selected from the group consisting of oligonucleotides which include a fragment of at least 10 contiguous nucleotides present in a nucleotide sequence as set forth in SEQ ID NO: 19 and at least one oligonucleotide selected from the group consisting of oligonucleotides which include a fragment of at least 10 contiguous nucleotides present in a nucleotide sequence as set forth in SEQ ID NO: 20; an oligonucleotide set including at least one oligonucleotide selected from the group consisting of oligonucleotides which include a fragment of at least 10 contiguous nucleotides present in a nucleotide sequence as set forth in SEQ ID NO: 21 and at least one oligonucleotide selected from the group consisting of oligonucleotides which include a fragment of at least 10 contiguous nucleotides present in a nucleotide sequence as set forth in SEQ ID NO: 22; an oligonucleotide set including at least one oligonucleotide selected from the group consisting of oligonucleotides which include a fragment of at least 10 contiguous nucleotides present in a nucleotide sequence as set forth in SEQ ID NO: 23 and at least one oligonucleotide selected from the group consisting of oligonucleotides which include a fragment of at least 10 contiguous nucleotides present in a nucleotide sequence as set forth in SEQ ID NO: 24; an oligonucleotide set including at least one oligonucleotide selected from the group consisting of oligonucleotides which include a fragment of at least 10 contiguous nucleotides present in a nucleotide sequence as set forth in SEQ ID NO: 25 and at least one oligonucleotide selected from the group consisting of oligonucleotides which include a fragment of at least 10 contiguous nucleotides present in a nucleotide sequence as set forth in SEQ ID NO: 26; an oligonucleotide set including at least one oligonucleotide selected from the group consisting of oligonucleotides which include a fragment of at least 10 contiguous nucleotides present in a nucleotide sequence as set forth in SEQ ID NO: 27 and at least one oligonucleotide selected from the group consisting of oligonucleotides which include a fragment of at least 10 contiguous nucleotides present in a nucleotide sequence as set forth in SEQ ID NO: 28; an oligonucleotide set including at least one oligonucleotide selected from the group consisting of oligonucleotides which include a fragment of at least 10 contiguous nucleotides present in a nucleotide sequence as set forth in SEQ ID NO: 29 and at least one oligonucleotide selected from the group consisting of oligonucleotides which include a fragment of at least 10 contiguous nucleotides present in a nucleotide sequence as set forth in SEQ ID NO: 30; an oligonucleotide set including at least one oligonucleotide selected from the group consisting of oligonucleotides which include a fragment of at least 10 contiguous nucleotides present in a nucleotide sequence as set forth in SEQ ID NO: 31 and at least one oligonucleotide selected from the group consisting of oligonucleotides which include a fragment of at least 10 contiguous nucleotides present in a nucleotide sequence as set forth in SEQ ID NO: 32; an oligonucleotide set including at least one oligonucleotide selected from the group consisting of oligonucleotides which include a fragment of at least 10 contiguous nucleotides present in a nucleotide sequence as set forth in SEQ ID NO: 33 and at least one oligonucleotide selected from the group consisting of oligonucleotides which include a fragment of at least 10 contiguous nucleotides present in a nucleotide sequence as set forth in SEQ ID NO: 34; an oligonucleotide set including at least one oligonucleotide selected from the group consisting of oligonucleotides which include a fragment of at least 10 contiguous nucleotides present in a nucleotide sequence as set forth in SEQ ID NO: 35 and at least one oligonucleotide selected from the group consisting of oligonucleotides which include a fragment of at least 10 contiguous nucleotides present in a nucleotide sequence as set forth in SEQ ID NO: 36; an oligonucleotide set including at least one oligonucleotide selected from the group consisting of oligonucleotides which include a fragment of at least 10 contiguous nucleotides present in a nucleotide sequence as set forth in SEQ ID NO: 37 and at least one oligonucleotide selected from the group consisting of oligonucleotides which include a fragment of at least 10 contiguous nucleotides present in a nucleotide sequence as set forth in SEQ ID NO: 38; an oligonucleotide set including at least one oligonucleotide selected from the group consisting of oligonucleotides which include a fragment of at least 10 contiguous nucleotides present in a nucleotide sequence as set forth in SEQ ID NO: 39 and at least one oligonucleotide selected from the group consisting of oligonucleotides which include a fragment of at least 10 contiguous nucleotides present in a nucleotide sequence as set forth in SEQ ID NO: 40; an oligonucleotide set including at least one oligonucleotide selected from the group consisting of oligonucleotides which include a fragment of at least 10 contiguous nucleotides present in a nucleotide sequence as set forth in SEQ ID NO: 41 and at least one oligonucleotide selected from the group consisting of oligonucleotides which include a fragment of at least 10 contiguous nucleotides present in a nucleotide sequence as set forth in SEQ ID NO: 42; an oligonucleotide set including at least one oligonucleotide selected from the group consisting of oligonucleotides which include a fragment of at least 10 contiguous nucleotides present in a nucleotide sequence as set forth in SEQ ID NO: 43 and at least one oligonucleotide selected from the group consisting of oligonucleotides which include a fragment of at least 10 contiguous nucleotides present in a nucleotide sequence as set forth in SEQ ID NO: 44; an oligonucleotide set including at least one oligonucleotide selected from the group consisting of oligonucleotides which include a fragment of at least 10 contiguous nucleotides present in a nucleotide sequence as set forth in SEQ ID NO: 45 and at least one oligonucleotide selected from the group consisting of oligonucleotides which include a fragment of at least 10 contiguous nucleotides present in a nucleotide sequence as set forth in SEQ ID NO: 46; an oligonucleotide set including at least one oligonucleotide selected from the group consisting of oligonucleotides which include a fragment of at least 10 contiguous nucleotides present in a nucleotide sequence as set forth in SEQ ID NO: 47 and at least one oligonucleotide selected from the group consisting of oligonucleotides which include a fragment of at least 10 contiguous nucleotides present in a nucleotide sequence as set forth in SEQ ID NO: 48; an oligonucleotide set including at least one oligonucleotide selected from the group consisting of oligonucleotides which include a fragment of at least 10 contiguous nucleotides present in a nucleotide sequence as set forth in SEQ ID NO: 49 and at least one oligonucleotide selected from the group consisting of oligonucleotides which include a fragment of at least 10 contiguous nucleotides present in a nucleotide sequence as set forth in SEQ ID NO: 50; and an oligonucleotide set including at least one oligonucleotide selected from the group consisting of oligonucleotides which include a fragment of at least 10 contiguous nucleotides present in a nucleotide sequence as set forth in SEQ ID NO: 51 and at least one oligonucleotide selected from the group consisting of oligonucleotides which include a fragment of at least 10 contiguous nucleotides present in a nucleotide sequence as set forth in SEQ ID NO: 52.

In the present invention, Spn represents Streptococcus pneumoniae, Pae represents Pseudomonas aeruginosa, Sau represents Staphylococcus aureus, Kpn represents Klebsiella pneumoniae, Aba represents Acinetobacter baumannii, Eco represents Escherichia coli, Ecl represents Enterobacter cloacae, and Eae represents Enterobacter aerogenes.

In the primer set of the present invention, the target sequence may be selected from a nucleotide region from position 425 to 890 of the aataph gene, a nucleotide region from position 343 to 722 of the ant gene, a nucleotide region from position 1618 to 2081 of the aph gene, a nucleotide region from position 256 to 449 of the CMY1 gene, a nucleotide region from position 508 to 738 of the CMY2 gene, a nucleotide region from position 55 to 571 of the CTX1 gene, a nucleotide region from position 346 to 688 of the CTX2 gene, a nucleotide region from position 630 to 1045 of the DHA gene, a nucleotide region from position 361 to 639 of the IMP gene, a nucleotide region from position 436 to 865 of the OXA gene, a nucleotide region from position 370 to 559 of the PER gene, a nucleotide region from position 116 to 336 of the SHV gene, a nucleotide region from position 425 to 783 of the TEM gene, a nucleotide region from position 572 to 848 of the VIM gene, a nucleotide region from position 138 to 597 of the ermA gene, a nucleotide region from position 127 to 390 of the ermB gene, a nucleotide region from position 40 to 290 of the ermC gene, a nucleotide region from position 46 to 288 of the mef gene, a nucleotide region from position 2933 to 3216 of the mecA gene, a nucleotide region from position 294 to 975 of the Spn pbp2b gene, a nucleotide region from position 399 to 703 of the Pae gyrA gene, a nucleotide region from position 164 to 317 of the Sau gyrA gene, a nucleotide region from position 38 to 497 of the Sau parC gene, a nucleotide region from position 1166 to 1501 of the Sau parE gene, a nucleotide region from position 106 to 442 of the vanA gene, and a nucleotide region from position 847 to 1045 of the vanB gene. Numbers used to represent a nucleotide region in the present invention represent positions counted from 5′ end of a nucleic acid.

The primer set of the present invention may be an oligonucleotide primer set for amplifying at least one target sequence selected from the aataph, ant, aph, CMY1, CMY2, CTX1, CTX2, DHA, IMP, OXA, PER, SHV, TEM, VIM, ermA, ermB, ermC, mef, mecA, Spn pbp2b, Pae gyrA, Sau gyrA, Sau parC, Sau parE, vanA, and vanB genes, which includes at least one oligonucleotide set selected from the group consisting of: an oligonucleotide set including an oligonucleotide having the nucleotide sequence as set forth in SEQ ID NO: 1 and an oligonucleotide having the nucleotide sequence as set forth in SEQ ID NO: 2; an oligonucleotide set including an oligonucleotide having the nucleotide sequence as set forth in SEQ ID NO: 3 and an oligonucleotide having the nucleotide sequence as set forth in SEQ ID NO: 4; an oligonucleotide set including an oligonucleotide having the nucleotide sequence as set forth in SEQ ID NO: 5 and an oligonucleotide having the nucleotide sequence as set forth in SEQ ID NO: 6; an oligonucleotide set including an oligonucleotide having the nucleotide sequence as set forth in SEQ ID NO: 7 and an oligonucleotide having the nucleotide sequence as set forth in SEQ ID NO: 8; an oligonucleotide set including an oligonucleotide having the nucleotide sequence as set forth in SEQ ID NO: 9 and an oligonucleotide having the nucleotide sequence as set forth in SEQ ID NO: 10; an oligonucleotide set including an oligonucleotide having the nucleotide sequence as set forth in SEQ ID NO: 11 and an oligonucleotide having the nucleotide sequence as set forth in SEQ ID NO: 12; an oligonucleotide set including an oligonucleotide having the nucleotide sequence as set forth in SEQ ID NO: 13 and an oligonucleotide having the nucleotide sequence as set forth in SEQ ID NO: 14; an oligonucleotide set including an oligonucleotide having the nucleotide sequence as set forth in SEQ ID NO: 15 and an oligonucleotide having the nucleotide sequence as set forth in SEQ ID NO: 16; an oligonucleotide set including an oligonucleotide having the nucleotide sequence as set forth in SEQ ID NO: 17 and an oligonucleotide having the nucleotide sequence as set forth in SEQ ID NO: 18; an oligonucleotide set including an oligonucleotide having the nucleotide sequence as set forth in SEQ ID NO: 19 and an oligonucleotide having the nucleotide sequence as set forth in SEQ ID NO: 20; an oligonucleotide set including an oligonucleotide having the nucleotide sequence as set forth in SEQ ID NO: 21 and an oligonucleotide having the nucleotide sequence as set forth in SEQ ID NO: 22; an oligonucleotide set including an oligonucleotide having the nucleotide sequence as set forth in SEQ ID NO: 23 and an oligonucleotide having the nucleotide sequence as set forth in SEQ ID NO: 24; an oligonucleotide set including an oligonucleotide having the nucleotide sequence as set forth in SEQ ID NO: 25 and an oligonucleotide having the nucleotide sequence as set forth in SEQ ID NO: 26; an oligonucleotide set including an oligonucleotide having the nucleotide sequence as set forth in SEQ ID NO: 27 and an oligonucleotide having the nucleotide sequence as set forth in SEQ ID NO: 28; an oligonucleotide set including an oligonucleotide having the nucleotide sequence as set forth in SEQ ID NO: 29 and an oligonucleotide having the nucleotide sequence as set forth in SEQ ID NO: 30; an oligonucleotide set including an oligonucleotide having the nucleotide sequence as set forth in SEQ ID NO: 31 and an oligonucleotide having the nucleotide sequence as set forth in SEQ ID NO: 32; an oligonucleotide set including an oligonucleotide having the nucleotide sequence as set forth in SEQ ID NO: 33 and an oligonucleotide having the nucleotide sequence as set forth in SEQ ID NO: 34; an oligonucleotide set including an oligonucleotide having the nucleotide sequence as set forth in SEQ ID NO: 35 and an oligonucleotide having the nucleotide sequence as set forth in SEQ ID NO: 36; an oligonucleotide set including an oligonucleotide having the nucleotide sequence as set forth in SEQ ID NO: 37 and an oligonucleotide having the nucleotide sequence as set forth in SEQ ID NO: 38; an oligonucleotide set including an oligonucleotide having the nucleotide sequence as set forth in SEQ ID NO: 39 and an oligonucleotide having the nucleotide sequence as set forth in SEQ ID NO: 40; an oligonucleotide set including an oligonucleotide having the nucleotide sequence as set forth in SEQ ID NO: 41 and an oligonucleotide having the nucleotide sequence as set forth in SEQ ID NO: 42; an oligonucleotide set including an oligonucleotide having the nucleotide sequence as set forth in SEQ ID NO: 43 and an oligonucleotide having the nucleotide sequence as set forth in SEQ ID NO: 44; an oligonucleotide set including an oligonucleotide having the nucleotide sequence as set forth in SEQ ID NO: 45 and an oligonucleotide having the nucleotide sequence as set forth in SEQ ID NO: 46; an oligonucleotide set including an oligonucleotide having the nucleotide sequence as set forth in SEQ ID NO: 47 and an oligonucleotide having the nucleotide sequence as set forth in SEQ ID NO: 48; an oligonucleotide set including an oligonucleotide having the nucleotide sequence as set forth in SEQ ID NO: 49 and an oligonucleotide having the nucleotide sequence as set forth in SEQ ID NO: 50; and an oligonucleotide set including an oligonucleotide having the nucleotide sequence as set forth in SEQ ID NO: 51 and an oligonucleotide having the nucleotide sequence as set forth in SEQ ID NO: 52.

The primer set of the present invention may be an oligonucleotide primer set for amplifying target sequences including the aataph, ant, aph, CMY1, CMY2, CTX1, CTX2, DHA, IMP, OXA, PER, SHV, TEM, VIM, ermA, ermB, ermC, mef, mecA, Spn pbp2b, Pae gyrA, Sau gyrA, Sau parC, Sau parE, vanA, and vanB genes, which includes: an oligonucleotide set including an oligonucleotide having the nucleotide sequence as set forth in SEQ ID NO: 1 and an oligonucleotide having the nucleotide sequence as set forth in SEQ ID NO: 2; an oligonucleotide set including an oligonucleotide having the nucleotide sequence as set forth in SEQ ID NO: 3 and an oligonucleotide having the nucleotide sequence as set forth in SEQ ID NO: 4; an oligonucleotide set including an oligonucleotide having the nucleotide sequence as set forth in SEQ ID NO: 5 and an oligonucleotide having the nucleotide sequence as set forth in SEQ ID NO: 6; an oligonucleotide set including an oligonucleotide having the nucleotide sequence as set forth in SEQ ID NO: 7 and an oligonucleotide having the nucleotide sequence as set forth in SEQ ID NO: 8; an oligonucleotide set including an oligonucleotide having the nucleotide sequence as set forth in SEQ ID NO: 9 and an oligonucleotide having the nucleotide sequence as set forth in SEQ ID NO: 10; an oligonucleotide set including an oligonucleotide having the nucleotide sequence as set forth in SEQ ID NO: 11 and an oligonucleotide having the nucleotide sequence as set forth in SEQ ID NO: 12; an oligonucleotide set including an oligonucleotide having the nucleotide sequence as set forth in SEQ ID NO: 13 and an oligonucleotide having the nucleotide sequence as set forth in SEQ ID NO: 14; an oligonucleotide set including an oligonucleotide having the nucleotide sequence as set forth in SEQ ID NO: 15 and an oligonucleotide having the nucleotide sequence as set forth in SEQ ID NO: 16; an oligonucleotide set including an oligonucleotide having the nucleotide sequence as set forth in SEQ ID NO: 17 and an oligonucleotide having the nucleotide sequence as set forth in SEQ ID NO: 18; an oligonucleotide set including an oligonucleotide having the nucleotide sequence as set forth in SEQ ID NO: 19 and an oligonucleotide having the nucleotide sequence as set forth in SEQ ID NO: 20; an oligonucleotide set including an oligonucleotide having the nucleotide sequence as set forth in SEQ ID NO: 21 and an oligonucleotide having the nucleotide sequence as set forth in SEQ ID NO: 22; an oligonucleotide set including an oligonucleotide having the nucleotide sequence as set forth in SEQ ID NO: 23 and an oligonucleotide having the nucleotide sequence as set forth in SEQ ID NO: 24; an oligonucleotide set including an oligonucleotide having the nucleotide sequence as set forth in SEQ ID NO: 25 and an oligonucleotide having the nucleotide sequence as set forth in SEQ ID NO: 26; an oligonucleotide set including an oligonucleotide having the nucleotide sequence as set forth in SEQ ID NO: 27 and an oligonucleotide having the nucleotide sequence as set forth in SEQ ID NO: 28; an oligonucleotide set including an oligonucleotide having the nucleotide sequence as set forth in SEQ ID NO: 29 and an oligonucleotide having the nucleotide sequence as set forth in SEQ ID NO: 30; an oligonucleotide set including an oligonucleotide having the nucleotide sequence as set forth in SEQ ID NO: 31 and an oligonucleotide having the nucleotide sequence as set forth in SEQ ID NO: 32; an oligonucleotide set including an oligonucleotide having the nucleotide sequence as set forth in SEQ ID NO: 33 and an oligonucleotide having the nucleotide sequence as set forth in SEQ ID NO: 34; an oligonucleotide set including an oligonucleotide having the nucleotide sequence as set forth in SEQ ID NO: 35 and an oligonucleotide having the nucleotide sequence as set forth in SEQ ID NO: 36; an oligonucleotide set including an oligonucleotide having the nucleotide sequence as set forth in SEQ ID NO: 37 and an oligonucleotide having the nucleotide sequence as set forth in SEQ ID NO: 38; an oligonucleotide set including an oligonucleotide having the nucleotide sequence as set forth in SEQ ID NO: 39 and an oligonucleotide having the nucleotide sequence as set forth in SEQ ID NO: 40; an oligonucleotide set including an oligonucleotide having the nucleotide sequence as set forth in SEQ ID NO: 41 and an oligonucleotide having the nucleotide sequence as set forth in SEQ ID NO: 42; an oligonucleotide set including an oligonucleotide having the nucleotide sequence as set forth in SEQ ID NO: 43 and an oligonucleotide having the nucleotide sequence as set forth in SEQ ID NO: 44; an oligonucleotide set including an oligonucleotide having the nucleotide sequence as set forth in SEQ ID NO: 45 and an oligonucleotide having the nucleotide sequence as set forth in SEQ ID NO: 46; an oligonucleotide set including an oligonucleotide having the nucleotide sequence as set forth in SEQ ID NO: 47 and an oligonucleotide having the nucleotide sequence as set forth in SEQ ID NO: 48; an oligonucleotide set including an oligonucleotide having the nucleotide sequence as set forth in SEQ ID NO: 49 and an oligonucleotide having the nucleotide sequence as set forth in SEQ ID NO: 50; and an oligonucleotide set including an oligonucleotide having the nucleotide sequence as set forth in SEQ ID NO: 51 and an oligonucleotide having the nucleotide sequence as set forth in SEQ ID NO: 52.

The primer set of the present invention was designed from predetermined regions of antibiotic resistance genes in antibiotic-resistant bacteria. Examples of the antibiotic-resistant bacteria include Spn, Sau, Kpn, Mca, Hin, Kpn, Eco, Pae, Mpn, Cpn, and Lpn. However, the antibiotic-resistant bacterial species are not limited to the above examples since the antibiotic resistance genes can be transferred from one species to another species, and thus, bacteria having the antibiotic resistance genes introduced therein have resistance against antibiotics. Commonly known antibiotic-resistant bacterial species and antibiotic resistance genes expressed in the bacterial species are summarized in Tables 1 and 2 below.

TABLE 1

| Antibiotic-resistant | Antibiotics | | |
|---|---|---|---|
| bacterial species | Sensitive | Resistant | Remarks |
| Spn | Penicillins, carbaphenems, | Aminoglycosides, novel | Increasing resistance to |
| | third generation | quinolones (some) | penicillin |
| | cepha-based, vancomycins | | |
| Methicillin-sensitive Sau | Penicillins, carbaphenems, | Old quinolones, third | Regarding macrolides, there are |
| | vancomycins, macrolides, | generation cepha-based, | bacterial species having |
| | aminoglycosides | monolactams | erythromycin-induced |
| | | | high-level resistance |
| Methicilllin-resistant | Vancomycins, Arbekacin, | Beta-lactams, macrolides, | Many minocycline/carbaphenem |
| Sau(MRSA) | rifampicins (partially | aminoglycosides | resistant bacterial species |
| | high-level tolerance) | | |
| Moraxella catarrhalis | Novel quinolones, | Penicillin G class | Beta lactamase-producing |
| | carbaphenems, macrolides, | | bacterial species (about 90%) |
| | beta-lactam combined with | | |
| | beta-lactamase inhibitor | | |
| Hin | Penicillins, novel quinolones, | Macrolides | Beta lactamase-producing |
| | second and third generation | | bacterial species (about 15%) |
| | cepha-based, | | |
| | amoxicillins/clavulanates | | |
| Kpn | Penicillins, novel quinolones, | Penicillins, macrolides, | Production of penicillinase, |
| | aminoglycosides | tetracyclines | resistance to penicillin |
| | (gentamycin etc.) | | |
| Eco | Cephenems, carbaphenems, | Macrolides | — |
| | novel quinolones, | | |
| | gentamycins | | |
| Pae | Piperacillins, cephtazidims, | Macrolides, ampicillins, | A limited number of antibiotics |
| | gentamycins, novel | tetracyclines | exhibit activity against bacterial |
| | quinolones | | species |
| Mpn | Tetracyclines, macrolides, | Beta-lactams | — |
| | novel quinolones (some) | | |
| Cpn | Tetracyclines, macrolides, | Beta-lactams, | — |
| | novel quinolones (some) | aminoglycosides | |
| Lpn | Macrolides (erythromycin), | Beta-lactams, | — |
| | tetracyclines, rifampicins | aminoglycosides | |

TABLE 2

| | | Antibiotic | | | |
|---|---|---|---|---|---|
| | Molecular | resistant | Target | | |
| Antibiotics | detection | bacteria | Gene(s) | Frequency | Reference |
| Aminoglycosides | Presence of | Sau, | aat/aph | 78% | J Korean Med. Sci 2003; 18: 631-6 |
| | gene | Spn, | ant | 45% | |
| | | Kpn, | aph | 50% | |
| | | Pae, | | | |
| | | Aba, | | | |
| | | Eco, | | | |
| | | Ecl | | | |
| | | Eae | | | |
| Beta- | Presence of | Kpn, | CMY-1, | Occurrence | J. of antimicrobial |
| Lactams | gene | Pae, | CMY-2, | frequency | Chemotherapy(2004) 54, 634-639, |
| | | Aba, | CTX-1, | (domestic) 100% | FEMS Microbiology letters |
| | | Eco, | CTX-2, | | 245(2005) 93-98 |
| | | Ecl, | IMP, | | |
| | | Eae | OXA, | | |
| | | | PER, | | |
| | | | SHV, | | |
| | | | TEM, | | |
| | | | VIM, | | |
| | | | DHA | | |
| Quinolones | Change of | Sau | gyrA | 98% (Pae),95% (Sau) | Antimicrobial agents and |
| | amino acid | Kpn | parC | 86% (Sau) | Chemotherapy February 1999, p. 406-409 |
| | | Pae | parE | 71% (Sau) | |
| Methicillins | Presence of | Sau | mecA | 98% | |
| | gene | | | | |
| Penicillins | Change of | Spn | PBP2b | 99% | J. clin. Microbiol. 34: 592-596 |
| | amino acid | | | | |
| Vancomycins | Pesence of | Sau, | VanA, | 100% | |
| | gene | Ecl | VanB | | |
| | | Eae | | | |
| Erythromycins | Presence of | Sau, | ermA, ermB, | 100% | |
| | gene | Ecl, | ermC, mef | | |
| | | Eae | | | |

Antibiotic resistance-determining genes presented in Tables 1 and 2, i.e., the aataph, ant, aph, CMY1, CMY2, CTX1, CTX2, DHA, IMP, OXA, PER, SHV, TEM, VIM, ermA, ermB, ermC, mef, mecA, Spn pbp2b, Pae gyrA, Sau gyrA, Sau parC, Sau parE, vanA, and vanB genes may have nucleotide sequences as set forth SEQ ID NOS: 156, 157, 158, 159, 160, 161, 162, 163, 164, 165, 166, 167, 168, 169, 170, 171, 172, 173, 174, 175, 176, 177, 178, 179, 180, and 181, respectively. The genes having the nucleotide sequences as set forth in SEQ ID NOS: 156-181 are consensus sequences of various genes having the same functions.

When performing PCR using the primer set of the present invention, a target sequence region sought to be amplified may be selected from the nucleotide region from position 425 to 890 of the aataph gene having the nucleotide sequence as set forth in SEQ ID NO: 156, the nucleotide region from position 343 to 722 of the ant gene having the nucleotide sequence as set forth in SEQ ID NO: 157, the nucleotide region from position 1618 to 2081 of the aph gene having the nucleotide sequence as set forth in SEQ ID NO: 158, the nucleotide region from position 256 to 449 of the CMY1 gene having the nucleotide sequence as set forth in SEQ ID NO: 159, the nucleotide region from position 508 to 738 of the CMY2 gene having the nucleotide sequence as set forth in SEQ ID NO: 160, the nucleotide region from position 55 to 571 of the CTX1 gene having the nucleotide sequence as set forth in SEQ ID NO: 161, the nucleotide region from position 346 to 688 of the CTX2 gene having the nucleotide sequence as set forth in SEQ ID NO: 162, the nucleotide region from position 630 to 1045 of the DHA gene having the nucleotide sequence as set forth in SEQ ID NO: 163, the nucleotide region from position 361 to 639 of the IMP gene having the nucleotide sequence as set forth in SEQ ID NO: 164, the nucleotide region from position 436 to 865 of the OXA gene having the nucleotide sequence as set forth in SEQ ID NO: 165, the nucleotide region from position 370 to 559 of the PER gene having the nucleotide sequence as set forth in SEQ ID NO: 166, the nucleotide region from position 116 to 336 of the SHV gene having the nucleotide sequence as set forth in SEQ ID NO: 167, the nucleotide region from position 425 to 783 of the TEM gene having the nucleotide sequence as set forth in SEQ ID NO: 168, the nucleotide region from position 572 to 848 of the VIM gene having the nucleotide sequence as set forth in SEQ ID NO: 169, the nucleotide region from position 138 to 597 of the ermA gene having the nucleotide sequence as set forth in SEQ ID NO: 170, the nucleotide region from position 127 to 390 of the ermB gene having the nucleotide sequence as set forth in SEQ ID NO: 171, the nucleotide region from position 40 to 290 of the ermC gene having the nucleotide sequence as set forth in SEQ ID NO: 172, the nucleotide region from position 46 to 288 of the mef gene having the nucleotide sequence as set forth in SEQ ID NO: 173, the nucleotide region from position 2933 to 3216 of the mecA gene having the nucleotide sequence as set forth in SEQ ID NO: 174, the nucleotide region from position 294 to 975 of the Spn pbp2b gene having the nucleotide sequence as set forth in SEQ ID NO: 175, the nucleotide region from position 399 to 703 of the Pae gyrA gene having the nucleotide sequence as set forth in SEQ ID NO: 176, the nucleotide region from position 164 to 317 of the Sau gyrA gene having the nucleotide sequence as set forth in SEQ ID NO: 177, the nucleotide region from position 38 to 497 of the Sau parC gene having the nucleotide sequence as set forth in SEQ ID NO: 178, the nucleotide region from position 1166 to 1501 of the Sau parE gene having the nucleotide sequence as set forth in SEQ ID NO: 179, the nucleotide region from position 106 to 442 of the vanA gene having the nucleotide sequence as set forth in SEQ ID NO: 180, and the nucleotide region from position 847 to 1045 of the vanB gene having the nucleotide sequence as set forth in SEQ ID NO: 181.

Reaction mechanisms according to the type of antibiotics are as follows.

TABLE 3

| Antibiotics | Reaction mechanism | Major resistance mechanism |
|---|---|---|
| Beta-lactams | PBP (peptidoglycan synthesis) | Beta lactamase |
| | inactivation | Low affinity PBP |
| | | Reduced transportation |
| Glycopeptides | Binding to peptidoglycan precursor | Precursor deformation |
| Aminoglycosides | Protein synthesis inhibition | Modifying enzyme (adenyl or PO4 |
| | (binding to 30S subunit) | addition) |
| Macrolides | Protein synthesis inhibition | rRNA methylation |
| | (binding to 30S subunit) | Efflux pumps |
| Quinolones | Topoisomerase inhibition (DNA | Modified target enzymes |
| | synthesis) | Efflux pumps |

Aminoglycoside-based antibiotics include amikacin. Beta-lactam-based antibiotics include cefaclor, cefprozil, cefuroxime, cefixime, cefotaxime, cefpodoxine, ceftazidime, ceftizoxime, ceftriaxone, cefepime, imipenem-cilastatin, meropenem, aztreonam, penicillin, etc. Quinolone-based antibiotics include ciprofloxacin, gatifloxacin, gemifloxacin, levofloxacin, moxifloxacin, norfloxacin, and ofloxacin. Erythromycin-based antibiotics include erythromycin. Vancomycin-based antibiotics include vancomycin.

The primer set of the present invention was designed from target sequences of antibiotic resistance-encoding genes expressed in the 11 antibiotic-resistant bacterial species, i.e., Spn, Sau, Kpn, Mca, Hin, Kpn, Eco, Pae, Mpn, Cpn, and Lpn. A primer set according to an exemplary embodiment of the present invention and target sequence regions amplified using the primer set are presented in Table 4 below.

TABLE 4
a primer set according to an exemplary embodiment
of the present invention and target sequence
regions amplified using the primer set

| Antibiotic | Primer | |
|---|---|---|
| resistance | (SEQ ID | |
| gene | NO:) | Amplification region |
| aataph | 1 | Nucleotide region from position 425 to 890 |
| | 2 | |
| ant | 3 | Nucleotide region from position 343 to 722 |
| | 4 | |
| aph | 5 | Nucleotide region from position 1618 to 2081 |
| | 6 | |
| CMY1 | 7 | Nucleotide region from position 256 to 449 |
| | 8 | |
| CMY2 | 9 | Nucleotide region from position 508 to 738 |
| | 10 | |
| CTX1 | 11 | Nucleotide region from position 55 to 571 |
| | 12 | |
| CTX2 | 13 | Nucleotide region from position 346 to 688 |
| | 14 | |
| DHA | 15 | Nucleotide region from position 630 to 1045 |
| | 16 | |
| IMP | 17 | Nucleotide region from position 361 to 639 |
| | 18 | |
| OXA | 19 | Nucleotide region from position 436 to 865 |
| | 20 | |
| PER | 21 | Nucleotide region from position 370 to 559 |
| | 22 | |
| SHV | 23 | Nucleotide region from position 116 to 336 |
| | 24 | |
| TEM | 25 | Nucleotide region from position 425 to 783 |
| | 26 | |
| VIM | 27 | Nucleotide region from position 572 to 848 |
| | 28 | |
| ermA | 29 | Nucleotide region from position 138 to 597 |
| | 30 | |
| ermB | 31 | Nucleotide region from position 127 to 390 |
| | 32 | |
| ermC | 33 | Nucleotide region from position 40 to 290 |
| | 34 | |
| Mef | 35 | Nucleotide region from position 46 to 288 |
| | 36 | |
| mecA | 37 | Nucleotide region from position 2933 to 3216 |
| | 38 | |
| Spn pbp2b | 39 | Nucleotide region from position 294 to 975 |
| | 40 | |
| Pae gyrA | 41 | Nucleotide region from position 399 to 703 |
| | 42 | |
| Sau gyrA | 43 | Nucleotide region from position 164 to 317 |
| | 44 | |
| Sau parC | 45 | Nucleotide region from position 38 to 497 |
| | 46 | |
| Sau parE | 47 | Nucleotide region from position 1166 to 1501 |
| | 48 | |
| vanA | 49 | Nucleotide region from position 106 to 442 |
| | 50 | |
| vanB | 51 | Nucleotide region from position 847 to 1045 |
| | 52 | |

The present invention also provides an oligonucleotide probe or probe set for detecting the presence or absence of at least one target sequence encoding antibiotic resistance activity selected from the group consisting of aataph, ant, aph, CMY1, CMY2, CTX1, CTX2, DHA, IMP, OXA, PER, SHV, TEM, VIM, ermA, ermB, ermC, mef, mecA, Spn wild-type pbp2b, Pae wild-type gyrA, Sau wild-type gyrA, Sau wild-type parC, Sau wild-type parE, vanA, and vanB genes, the oligonucleotide probe or probe set being selected from the group consisting of:

an oligonucleotide probe capable of hybridizing with a nucleotide region from position 425 to 890 of the aataph gene, including at least one oligonucleotide selected from the group consisting of oligonucleotides which include a fragment of at least 10 contiguous nucleotides present in at least one nucleotide sequence selected from the group consisting of nucleotide sequences as set forth in SEQ ID NOS: 53-55 and complementary oligonucleotides thereof;

an oligonucleotide probe capable of hybridizing with a nucleotide region from position 343 to 722 of the ant gene, including at least one oligonucleotide selected from the group consisting of oligonucleotides which include a fragment of at least 10 contiguous nucleotides present in at least one nucleotide sequence selected from the group consisting of nucleotide sequences as set forth in SEQ ID NOS: 56-57 and complementary oligonucleotides thereof;

an oligonucleotide probe capable of hybridizing with a nucleotide region from position 1618 to 2081 of the aph gene, including at least one oligonucleotide selected from the group consisting of oligonucleotides which include a fragment of at least 10 contiguous nucleotides present in at least one nucleotide sequence selected from the group consisting of nucleotide sequences as set forth in SEQ ID NOS: 58-59 and complementary oligonucleotides thereof;

an oligonucleotide probe capable of hybridizing with a nucleotide region from position 256 to 449 of the CMY1 gene, including at least one oligonucleotide selected from the group consisting of oligonucleotides which include a fragment of at least 10 contiguous nucleotides present in at least one nucleotide sequence selected from the group consisting of nucleotide sequences as set forth in SEQ ID NOS: 60 to 61 and complementary oligonucleotides thereof;

an oligonucleotide probe capable of hybridizing with a nucleotide region from position 508 to 738 of the CMY2 gene, including at least one oligonucleotide selected from the group consisting of oligonucleotides which include a fragment of at least 10 contiguous nucleotides present in at least one nucleotide sequence selected from the group consisting of nucleotide sequences as set forth in SEQ ID NOS: 62-64 and complementary oligonucleotides thereof;

an oligonucleotide probe capable of hybridizing with a nucleotide region from position 55 to 571 of the CTX1 gene, including at least one oligonucleotide selected from the group consisting of oligonucleotides which include a fragment of at least 10 contiguous nucleotides present in at least one nucleotide sequence selected from the group consisting of nucleotide sequences as set forth in SEQ ID NOS: 65-66 and complementary oligonucleotides thereof;

an oligonucleotide probe capable of hybridizing with a nucleotide region from position 346 to 688 of the CTX2 gene, including at least one oligonucleotide selected from the group consisting of oligonucleotides which include a fragment of at least 10 contiguous nucleotides present in at least one nucleotide sequence selected from the group consisting of nucleotide sequences as set forth in SEQ ID NOS: 67-68 and complementary oligonucleotides thereof;

an oligonucleotide probe capable of hybridizing with a nucleotide region from position 630 to 1045 of the DHA gene, including at least one oligonucleotide selected from the group consisting of oligonucleotides which include a fragment of at least 10 contiguous nucleotides present in at least one nucleotide sequence selected from the group consisting of nucleotide sequences as set forth in SEQ ID NOS: 69-70 and complementary oligonucleotides thereof;

an oligonucleotide probe capable of hybridizing with a nucleotide region from position 361 to 639 of the IMP gene, including at least one oligonucleotide selected from the group consisting of oligonucleotides which include a fragment of at least 10 contiguous nucleotides present in at least one nucleotide sequence selected from the group consisting of nucleotide sequences as set forth in SEQ ID NOS: 71-73 and complementary oligonucleotides thereof;

an oligonucleotide probe capable of hybridizing with a nucleotide region from position 436 to 865 of the OXA gene, including at least one oligonucleotide selected from the group consisting of oligonucleotides which include a fragment of at least 10 contiguous nucleotides present in at least one nucleotide sequence selected from the group consisting of nucleotide sequences as set forth in SEQ ID NOS: 74-75 and complementary oligonucleotides thereof;

an oligonucleotide probe capable of hybridizing with a nucleotide region from position 370 to 559 of the PER gene, including at least one oligonucleotide selected from the group consisting of oligonucleotides which include a fragment of at least 10 contiguous nucleotides present in at least one nucleotide sequence selected from the group consisting of nucleotide sequences as set forth in SEQ ID NOS: 76-77 and complementary oligonucleotides thereof;

an oligonucleotide probe capable of hybridizing with a nucleotide region from position 116 to 336 of the SHV gene, including at least one oligonucleotide selected from the group consisting of oligonucleotides which include a fragment of at least 10 contiguous nucleotides present in at least one nucleotide sequence selected from the group consisting of nucleotide sequences as set forth in SEQ ID NOS: 78-79 and complementary oligonucleotides thereof;

an oligonucleotide probe capable of hybridizing with a nucleotide region from position 425 to 783 of the TEM gene, including at least one oligonucleotide selected from the group consisting of oligonucleotides which include a fragment of at least 10 contiguous nucleotides present in at least one nucleotide sequence selected from the group consisting of nucleotide sequences as set forth in SEQ ID NOS: 80-81 and complementary oligonucleotides thereof;

an oligonucleotide probe capable of hybridizing with a nucleotide region from position 572 to 848 of the VIM gene, including at least one oligonucleotide selected from the group consisting of oligonucleotides which include a fragment of at least 10 contiguous nucleotides present in at least one nucleotide sequence selected from the group consisting of nucleotide sequences as set forth in SEQ ID NOS: 82-83 and complementary oligonucleotides thereof;

an oligonucleotide probe capable of hybridizing with a nucleotide region from position 138 to 597 of the ermA gene, including at least one oligonucleotide selected from the group consisting of oligonucleotides which include a fragment of at least 10 contiguous nucleotides present in at least one nucleotide sequence selected from the group consisting of nucleotide sequences as set forth in SEQ ID NOS: 84-85 and complementary oligonucleotides thereof;

an oligonucleotide probe capable of hybridizing with a nucleotide region from position 127 to 390 of the ermB gene, including at least one oligonucleotide selected from the group consisting of oligonucleotides which include a fragment of at least 10 contiguous nucleotides present in at least one nucleotide sequence selected from the group consisting of nucleotide sequences as set forth in SEQ ID NOS: 86-87 and complementary oligonucleotides thereof;

an oligonucleotide probe capable of hybridizing with a nucleotide region from position 40 to 290 of the ermC gene, including at least one oligonucleotide selected from the group consisting of oligonucleotides which include a fragment of at least 10 contiguous nucleotides present in at least one nucleotide sequence selected from the group consisting of nucleotide sequences as set forth in SEQ ID NOS: 88-92 and complementary oligonucleotides thereof;

an oligonucleotide probe capable of hybridizing with a nucleotide region from position 46 to 288 of the mef gene, including at least one oligonucleotide selected from the group consisting of oligonucleotides which include a fragment of at least 10 contiguous nucleotides present in at least one nucleotide sequence selected from the group consisting of nucleotide sequences as set forth in SEQ ID NOS: 93-95 and complementary oligonucleotides thereof;

an oligonucleotide probe capable of hybridizing with a nucleotide region from position 2933 to 3216 of the mecA gene, including at least one oligonucleotide selected from the group consisting of oligonucleotides which include a fragment of at least 10 contiguous nucleotides present in at least one nucleotide sequence selected from the group consisting of nucleotide sequences as set forth in SEQ ID NOS: 96-101 and complementary oligonucleotides thereof;

an oligonucleotide probe capable of hybridizing with a nucleotide region from position 106 to 442 of the vanA gene, including at least one oligonucleotide selected from the group consisting of oligonucleotides which include a fragment of at least 10 contiguous nucleotides present in at least one nucleotide sequence selected from the group consisting of nucleotide sequences as set forth in SEQ ID NOS: 102-103 and complementary oligonucleotides thereof;

an oligonucleotide probe capable of hybridizing with a nucleotide region from position 847 to 1045 of the vanB gene, including at least one oligonucleotide selected from the group consisting of oligonucleotides which include a fragment of at least 10 contiguous nucleotides present in at least one nucleotide sequence selected from the group consisting of nucleotide sequences as set forth in SEQ ID NOS: 104-105 and complementary oligonucleotides thereof;

an oligonucleotide probe capable of hybridizing with a nucleotide region from position 399 to 703 of the Pae wild-type gyrA gene, including at least one oligonucleotide selected from the group consisting of oligonucleotides which include a fragment of at least 10 contiguous nucleotides present in at least one nucleotide sequence selected from the group consisting of nucleotide sequences as set forth in SEQ ID NOS: 106, 108, 110, 112, 114, 116, 118, 120, and 122, and complementary oligonucleotides thereof;

an oligonucleotide probe capable of hybridizing with a nucleotide region from position 164 to 317 of the Sau wild-type gyrA gene, including at least one oligonucleotide selected from the group consisting of oligonucleotides which include a fragment of at least 10 contiguous nucleotides present in at least one nucleotide sequence selected from the group consisting of nucleotide sequences as set forth in SEQ ID NOS: 124, 126, 128, and 130, and complementary oligonucleotides thereof;

an oligonucleotide probe capable of hybridizing with a nucleotide region from position 38 to 497 of the Sau wild-type parC gene, including at least one oligonucleotide selected from the group consisting of oligonucleotides which include a fragment of at least 10 contiguous nucleotides present in at least one nucleotide sequence selected from the group consisting of nucleotide sequences as set forth in SEQ ID NOS: 132, 134, and 136, and complementary oligonucleotides thereof;

an oligonucleotide probe capable of hybridizing with a nucleotide region from position 1166 to 1501 of the Sau wild-type parE gene, including at least one oligonucleotide selected from the group consisting of oligonucleotides which include a fragment of at least 10 contiguous nucleotides present in at least one nucleotide sequence selected from the group consisting of nucleotide sequences as set forth in SEQ ID NOS: 138 and 140 and complementary oligonucleotides thereof; and

an oligonucleotide probe capable of hybridizing with a nucleotide region from position 294 to 975 of the Spn wild-type pbp2b gene, including at least one oligonucleotide selected from the group consisting of oligonucleotides which include a fragment of at least 10 contiguous nucleotides present in at least one nucleotide sequence selected from the group consisting of nucleotide sequences as set forth in SEQ ID NOS: 142, 144, 146, 148, and 150, and complementary oligonucleotides thereof.

The probe or probe set of the present invention may be an oligonucleotide probe or probe set for detecting the presence or absence of at least one target sequence encoding antibiotic resistance activity selected from the aataph, ant, aph, CMY1, CMY2, CTX1, CTX2, DHA, IMP, OXA, PER, SHV, TEM, VIM, ermA, ermB, ermC, mef, mecA, Spn wild-type pbp2b, Pae wild-type gyrA, Sau wild-type gyrA, Sau wild-type parC, Sau wild-type parE, vanA, and vanB genes, the oligonucleotide probe or probe set being selected from the group consisting of:

an oligonucleotide probe capable of hybridizing with the nucleotide region from position 425 to 890 of the aataph gene, including at least one oligonucleotide selected from the group consisting of oligonucleotides having the nucleotide sequences as set forth in SEQ ID NOS: 53-55 and complementary oligonucleotides thereof;

an oligonucleotide probe capable of hybridizing with the nucleotide region from position 343 to 722 of the ant gene, including at least one oligonucleotide selected from the group consisting of oligonucleotides having the nucleotide sequences as set forth in SEQ ID NOS: 56-57 and complementary oligonucleotides thereof;

an oligonucleotide probe capable of hybridizing with the nucleotide region from position 1618 to 2081 of the aph gene, including at least one oligonucleotide selected from the group consisting of oligonucleotides having the nucleotide sequences as set forth in SEQ ID NOS: 58-59 and complementary oligonucleotides thereof;

an oligonucleotide probe capable of hybridizing with the nucleotide region from position 256 to 449 of the CMY1 gene, including at least one oligonucleotide selected from the group consisting of oligonucleotides having the nucleotide sequences as set forth in SEQ ID NOS: 60-61 and complementary oligonucleotides thereof;

an oligonucleotide probe capable of hybridizing with the nucleotide region from position 508 to 738 of the CMY2 gene, including at least one oligonucleotide selected from the group consisting of oligonucleotides having the nucleotide sequences as set forth in SEQ ID NOS: 62-64 and complementary oligonucleotides thereof;

an oligonucleotide probe capable of hybridizing with the nucleotide region from position 55 to 571 of the CTX1 gene, including at least one oligonucleotide selected from the group consisting of oligonucleotides having the nucleotide sequences as set forth in SEQ ID NOS: 65-66 and complementary oligonucleotides thereof;

an oligonucleotide probe capable of hybridizing with the nucleotide region from position 346 to 688 of the CTX2 gene, including at least one oligonucleotide selected from the group consisting of oligonucleotides having the nucleotide sequences as set forth in SEQ ID NOS: 67-68 and complementary oligonucleotides thereof;

an oligonucleotide probe capable of hybridizing with the nucleotide region from position 630 to 1045 of the DHA gene, including at least one oligonucleotide selected from the group consisting of oligonucleotides having the nucleotide sequences as set forth in SEQ ID NOS: 69-70 and complementary oligonucleotides thereof;

an oligonucleotide probe capable of hybridizing with the nucleotide region from position 361 to 639 of the IMP gene, including at least one oligonucleotide selected from the group consisting of oligonucleotides having the nucleotide sequences as set forth in SEQ ID NOS: 71-73 and complementary oligonucleotides thereof;

an oligonucleotide probe capable of hybridizing with the nucleotide region from position 436 to 865 of the OXA gene, including at least one oligonucleotide selected from the group consisting of oligonucleotides having the nucleotide sequences as set forth in SEQ ID NOS: 74-75 and complementary oligonucleotides thereof;

an oligonucleotide probe capable of hybridizing with the nucleotide region from position 370 to 559 of the PER gene, including at least one oligonucleotide selected from the group consisting of oligonucleotides having the nucleotide sequences as set forth in SEQ ID NOS: 76-77 and complementary oligonucleotides thereof;

an oligonucleotide probe capable of hybridizing with the nucleotide region from position 116 to 336 of the SHV gene, including at least one oligonucleotide selected from the group consisting of oligonucleotides having the nucleotide sequences as set forth in SEQ ID NOS: 78-79 and complementary oligonucleotides thereof;

an oligonucleotide probe capable of hybridizing with the nucleotide region from position 425 to 783 of the TEM gene, including at least one oligonucleotide selected from the group consisting of oligonucleotides having the nucleotide sequences as set forth in SEQ ID NOS: 80-81 and complementary oligonucleotides thereof;

an oligonucleotide probe capable of hybridizing with the nucleotide region from position 572 to 848 of the VIM gene, including at least one oligonucleotide selected from the group consisting of oligonucleotides having the nucleotide sequences as set forth in SEQ ID NOS: 82-83 and complementary oligonucleotides thereof;

an oligonucleotide probe capable of hybridizing with the nucleotide region from position 138 to 597 of the ermA gene, including at least one oligonucleotide selected from the group consisting of oligonucleotides having the nucleotide sequences as set forth in SEQ ID NOS: 84-85 and complementary oligonucleotides thereof;

an oligonucleotide probe capable of hybridizing with the nucleotide region from position 127 to 390 of the ermB gene, including at least one oligonucleotide selected from the group consisting of oligonucleotides having the nucleotide sequences as set forth in SEQ ID NOS: 86-87 and complementary oligonucleotides thereof;

an oligonucleotide probe capable of hybridizing with the nucleotide region from position 40 to 290 of the ermC gene, including at least one oligonucleotide selected from the group consisting of oligonucleotides having the nucleotide sequences as set forth in SEQ ID NOS: 88-92 and complementary oligonucleotides thereof;

an oligonucleotide probe capable of hybridizing with the nucleotide region from position 46 to 288 of the mef gene, including at least one oligonucleotide selected from the group consisting of oligonucleotides having the nucleotide sequences as set forth in SEQ ID NOS: 93-95 and complementary oligonucleotides thereof;

an oligonucleotide probe capable of hybridizing with the nucleotide region from position 2933 to 3216 of the mecA gene, including at least one oligonucleotide selected from the group consisting of oligonucleotides having the nucleotide sequences as set forth in SEQ ID NOS: 96-101 and complementary oligonucleotides thereof;

an oligonucleotide probe capable of hybridizing with the nucleotide region from position 106 to 442 of the vanA gene, including at least one oligonucleotide selected from the group consisting of oligonucleotides having the nucleotide sequences as set forth in SEQ ID NOS: 102-103 and complementary oligonucleotides thereof;

an oligonucleotide probe capable of hybridizing with the nucleotide region from position 847 to 1045 of the vanB gene, including at least one oligonucleotide selected from the group consisting of oligonucleotides having the nucleotide sequences as set forth in SEQ ID NOS: 104-105 and complementary oligonucleotides thereof;

an oligonucleotide probe capable of hybridizing with the nucleotide region from position 399 to 703 of the Pae wild-type gyrA gene, including at least one oligonucleotide selected from the group consisting of oligonucleotides having the nucleotide sequences as set forth in SEQ ID NOS: 106, 108, 110, 112, 114, 116, 118, 120, and 122 and complementary oligonucleotides thereof;

an oligonucleotide probe capable of hybridizing with the nucleotide region from position 164 to 317 of the Sau wild-type gyrA gene, including at least one oligonucleotide selected from the group consisting of oligonucleotides having the nucleotide sequences as set forth in SEQ ID NOS: 124, 126, 128, and 130 and complementary oligonucleotides thereof;

an oligonucleotide probe capable of hybridizing with the nucleotide region from position 38 to 497 of the Sau wild-type parC gene, including at least one oligonucleotide selected from the group consisting of oligonucleotides having the nucleotide sequences as set forth in SEQ ID NOS: 132, 134, and 136 and complementary oligonucleotides thereof;

an oligonucleotide probe capable of hybridizing with the nucleotide region from position 1166 to 1501 of the Sau wild-type parE gene, including at least one oligonucleotide selected from the group consisting of oligonucleotides having the nucleotide sequences as set forth in SEQ ID NOS: 138 and 140 and complementary oligonucleotides thereof; and an oligonucleotide probe capable of hybridizing with the nucleotide region from position 294 to 975 of the Spn wild-type pbp2b gene, including at least one oligonucleotide selected from the group consisting of oligonucleotides having the nucleotide sequences as set forth in SEQ ID NOS: 142, 144, 146, 148, 150, 152, and 154 and complementary oligonucleotides thereof.

The probe or probe set of the present invention may be an oligonucleotide probe or probe set for detecting the presence or absence of at least one target sequence encoding antibiotic resistance activity selected from the aataph, ant, aph, CMY1, CMY2, CTX1, CTX2, DHA, IMP, OXA, PER, SHV, TEM, VIM, ermA, ermB, ermC, mef, mecA, Spn wild-type pbp2b, Pae wild-type gyrA, Sau wild-type gyrA, Sau wild-type parC, Sau wild-type parE, vanA, and vanB genes, the oligonucleotide probe or probe set being selected from the group consisting of:

oligonucleotide probes capable of hybridizing with the nucleotide region from position 425 to 890 of the aataph gene, including oligonucleotides having the nucleotide sequences as set forth in SEQ ID NOS: 53-55 or complementary oligonucleotides thereof;

oligonucleotide probes capable of hybridizing with the nucleotide region from position 343 to 722 of the ant gene, including oligonucleotides having the nucleotide sequences as set forth in SEQ ID NOS: 56-57 or complementary oligonucleotides thereof;

oligonucleotide probes capable of hybridizing with the nucleotide region from position 1618 to 2081 of the aph gene, including oligonucleotides having the nucleotide sequences as set forth in SEQ ID NOS: 58-59 or complementary oligonucleotides thereof;

oligonucleotide probes capable of hybridizing with the nucleotide region from position 256 to 449 of the CMY1 gene, including oligonucleotides having the nucleotide sequences as set forth in SEQ ID NOS: 60-61 or complementary oligonucleotides thereof;

oligonucleotide probes capable of hybridizing with the nucleotide region from position 508 to 738 of the CMY2 gene, including oligonucleotides having the nucleotide sequences as set forth in SEQ ID NOS: 62-64 or complementary oligonucleotides thereof;

oligonucleotide probes capable of hybridizing with the nucleotide region from position 55 to 571 of the CTX1 gene, including oligonucleotides having the nucleotide sequences as set forth in SEQ ID NOS: 65-66 or complementary oligonucleotides thereof;

oligonucleotide probes capable of hybridizing with the nucleotide region from position 346 to 688 of the CTX2 gene, including oligonucleotides having the nucleotide sequences as set forth in SEQ ID NOS: 67-68 or complementary oligonucleotides thereof;

oligonucleotide probes capable of hybridizing with the nucleotide region from position 630 to 1045 of the DHA gene, including oligonucleotides having the nucleotide sequences as set forth in SEQ ID NOS: 69-70 or complementary oligonucleotides thereof;

oligonucleotide probes capable of hybridizing with the nucleotide region from position 361 to 639 of the IMP gene, including oligonucleotides having the nucleotide sequences as set forth in SEQ ID NOS: 71-73 or complementary oligonucleotides thereof;

oligonucleotide probes capable of hybridizing with the nucleotide region from position 436 to 865 of the OXA gene, including oligonucleotides having the nucleotide sequences as set forth in SEQ ID NOS: 74-75 or complementary oligonucleotides thereof;

oligonucleotide probes capable of hybridizing with the nucleotide region from position 370 to 559 of the PER gene, including oligonucleotides having the nucleotide sequences as set forth in SEQ ID NOS: 76-77 or complementary oligonucleotides thereof;

oligonucleotide probes capable of hybridizing with the nucleotide region from position 116 to 336 of the SHV gene, including oligonucleotides having the nucleotide sequences as set forth in SEQ ID NOS: 78-79 or complementary oligonucleotides thereof;

oligonucleotide probes capable of hybridizing with the nucleotide region from position 425 to 783 of the TEM gene, including oligonucleotides having the nucleotide sequences as set forth in SEQ ID NOS: 80-81 or complementary oligonucleotides thereof;

oligonucleotide probes capable of hybridizing with the nucleotide region from position 572 to 848 of the VIM gene, including oligonucleotides having the nucleotide sequences as set forth in SEQ ID NOS: 82-83 or complementary oligonucleotides thereof;

oligonucleotide probes capable of hybridizing with the nucleotide region from position 138 to 597 of the ermA gene, including oligonucleotides having the nucleotide sequences as set forth in SEQ ID NOS: 84-85 or complementary oligonucleotides thereof;

oligonucleotide probes capable of hybridizing with the nucleotide region from position 127 to 390 of the ermB gene, including oligonucleotides having the nucleotide sequences as set forth in SEQ ID NOS: 86-87 or complementary oligonucleotides thereof;

oligonucleotide probes capable of hybridizing with the nucleotide region from position 40 to 290 of the ermC gene, including oligonucleotides having the nucleotide sequences as set forth in SEQ ID NOS: 88-92 or complementary oligonucleotides thereof;

oligonucleotide probes capable of hybridizing with the nucleotide region from position 46 to 288 of the mef gene, including oligonucleotides having the nucleotide sequences as set forth in SEQ ID NOS: 93-95 or complementary oligonucleotides thereof;

oligonucleotide probes capable of hybridizing with the nucleotide region from position 2933 to 3216 of the mecA gene, including oligonucleotides having the nucleotide sequences as set forth in SEQ ID NOS: 96-101 or complementary oligonucleotides thereof;

oligonucleotide probes capable of hybridizing with the nucleotide region from position 106 to 442 of the vanA gene, including oligonucleotides having the nucleotide sequences as set forth in SEQ ID NOS: 102-103 or complementary oligonucleotides thereof;

oligonucleotide probes capable of hybridizing with the nucleotide region from position 847 to 1045 of the vanB gene, including oligonucleotides having the nucleotide sequences as set forth in SEQ ID NOS: 104-105 or complementary oligonucleotides thereof;

oligonucleotide probes capable of hybridizing with the nucleotide region from position 399 to 703 of the Pae wild-type gyrA gene, including oligonucleotides having the nucleotide sequences as set forth in SEQ ID NOS: 106, 108, 110, 112, 114, 116, 118, 120, and 122, or complementary oligonucleotides thereof;

oligonucleotide probes capable of hybridizing with the nucleotide region from position 164 to 317 of the Sau wild-type gyrA gene, including oligonucleotides having the nucleotide sequences as set forth in SEQ ID NOS: 124, 126, 128, and 130, or complementary oligonucleotides thereof;

oligonucleotide probes capable of hybridizing with the nucleotide region from position 38 to 497 of the Sau wild-type parC gene, including oligonucleotides having the nucleotide sequences as set forth in SEQ ID NOS: 132, 134, and 136, or complementary oligonucleotides thereof;

oligonucleotide probes capable of hybridizing with the nucleotide region from position 1166 to 1501 of the Sau wild-type parE gene, including oligonucleotides having the nucleotide sequences as set forth in SEQ ID NOS: 138 and 140, or complementary oligonucleotides thereof; and

oligonucleotide probes capable of hybridizing with the nucleotide region from position 294 to 975 of the Spn wild-type pbp2b gene, including oligonucleotides having the nucleotide sequences as set forth in SEQ ID NOS: 142, 144, 146, 148, 150, 152, and 154, or complementary oligonucleotides thereof.

The probe or probe set of the present invention may further include an oligonucleotide probe or probe set capable of hybridizing with at least one antibiotic resistance-inactivated mutant gene selected from the group consisting of Pae mutant-type gyrA, Sau mutant-type gyrA, Sau mutant-type parC, Sau mutant-type parE, and Spn mutant-type pbp2b genes, the oligonucleotide probe or probe set being selected from the group consisting of:

an oligonucleotide probe capable of hybridizing with a nucleotide region from position 399 to 703 of the Pae mutant-type gyrA gene, including at least one oligonucleotide selected from the group consisting of oligonucleotides which include a fragment of at least 10 contiguous nucleotides present in at least one nucleotide sequence selected from the group consisting of nucleotide sequences as set forth in SEQ ID NOS: 107, 109, 111, 113, 115, 117, 119, 121, and 123, and complementary oligonucleotides thereof;

an oligonucleotide probe capable of hybridizing with a nucleotide region from position 164 to 317 of the Sau mutant-type gyrA gene, including at least one oligonucleotide selected from the group consisting of oligonucleotides which include a fragment of at least 10 contiguous nucleotides present in at least one nucleotide sequence selected from the group consisting of nucleotide sequences as set forth in SEQ ID NOS: 125, 127, 129, and 131, and complementary oligonucleotides thereof;

an oligonucleotide probe capable of hybridizing with a nucleotide region from position 38 to 497 of the Sau mutant-type parC gene, including at least one oligonucleotide selected from the group consisting of oligonucleotides which include a fragment of at least 10 contiguous nucleotides present in at least one nucleotide sequence selected from the group consisting of nucleotide sequences as set forth in SEQ ID NOS: 133, 135, and 137, and complementary oligonucleotides thereof;

an oligonucleotide probe capable of hybridizing with a nucleotide region from position 1166 to 1501 of the Sau mutant-type parE gene, including at least one oligonucleotide selected from the group consisting of oligonucleotides which include a fragment of at least 10 contiguous nucleotides present in at least one nucleotide sequence selected from the group consisting of nucleotide sequences as set forth in SEQ ID NOS: 139 and 141 and complementary oligonucleotides thereof; and

an oligonucleotide probe capable of hybridizing with a nucleotide region from position 294 to 975 of the Spn mutant-type pbp2b gene, including at least one oligonucleotide selected from the group consisting of oligonucleotides which include a fragment of at least 10 contiguous nucleotides present in at least one nucleotide sequence selected from the group consisting of nucleotide sequences as set forth in SEQ ID NOS: 143, 145, 147, 149, 151, 153, and 155, and complementary oligonucleotides thereof.

The probe or probe set of the present invention may further include an oligonucleotide probe or probe set capable of hybridizing with at least one antibiotic resistance gene selected from the group consisting of Pae mutant-type gyrA, Sau mutant-type gyrA, Sau mutant-type parC, Sau mutant-type parE, and Spn mutant-type pbp2b genes, the oligonucleotide probe or probe set being selected from the group consisting of:

an oligonucleotide probe capable of hybridizing with the nucleotide region from position 399 to 703 of the Pae mutant-type gyrA gene, including at least one oligonucleotide selected from the group consisting of oligonucleotides having the nucleotide sequences as set forth in SEQ ID NOS: 107, 109, 111, 113, 115, 117, 119, 121, and 123, and complementary oligonucleotides thereof;

an oligonucleotide probe capable of hybridizing with the nucleotide region from position 164 to 317 of the Sau mutant-type gyrA gene, including at least one oligonucleotide selected from the group consisting of oligonucleotides having the nucleotide sequences as set forth in SEQ ID NOS: 125, 127, 129, and 131, and complementary oligonucleotides thereof;

an oligonucleotide probe capable of hybridizing with the nucleotide region from position 38 to 497 of the Sau mutant-type parC gene, including at least one oligonucleotide selected from the group consisting of oligonucleotides having the nucleotide sequences as set forth in SEQ ID NOS: 133, 135, and 137, and complementary oligonucleotides thereof;

an oligonucleotide probe capable of hybridizing with the nucleotide region from position 1166 to 1501 of the Sau mutant-type parE gene, including at least one oligonucleotide selected from the group consisting of oligonucleotides having the nucleotide sequences as set forth in SEQ ID NOS: 139 and 141 and complementary oligonucleotides thereof; and

an oligonucleotide probe capable of hybridizing with the nucleotide region from position 294 to 975 of the Spn mutant-type pbp2b gene, including at least one oligonucleotide selected from the group consisting of oligonucleotides having the nucleotide sequences as set forth in SEQ ID NOS: 143, 145, 147, 149, 151, 153, and 155 and complementary oligonucleotides thereof.

The probe or probe set of the present invention may further include an oligonucleotide probe set capable of hybridizing with antibiotic resistance-inactivated mutant genes including Pae mutant-type gyrA, Sau mutant-type gyrA, Sau mutant-type parC, Sau mutant-type parE, and Spn mutant-type pbp2b genes, the oligonucleotide probe set being selected from the group consisting of:

oligonucleotide probes capable of hybridizing with the nucleotide region from position 399 to 703 of the Pae mutant-type gyrA gene, including oligonucleotides having the nucleotide sequences as set forth in SEQ ID NOS: 107, 109, 111, 113, 115, 117, 119, 121, and 123, or complementary oligonucleotides thereof;

oligonucleotide probes capable of hybridizing with the nucleotide region from position 164 to 317 of the Sau mutant-type gyrA gene, including oligonucleotides having the nucleotide sequences as set forth in SEQ ID NOS: 125, 127, 129, and 131, or complementary oligonucleotides thereof;

oligonucleotide probes capable of hybridizing with the nucleotide region from position 38 to 497 of the Sau mutant-type parC gene, including oligonucleotides having the nucleotide sequences as set forth in SEQ ID NOS: 133, 135, and 137, or complementary oligonucleotides thereof;

oligonucleotide probes capable of hybridizing with the nucleotide region from position 1166 to 1501 of the Sau mutant-type parE gene, including oligonucleotides having the nucleotide sequences as set forth in SEQ ID NOS: 139 and 141 or complementary oligonucleotides thereof; and

oligonucleotide probes capable of hybridizing with the nucleotide region from position 294 to 975 of the Spn mutant-type pbp2b gene, including oligonucleotides having the nucleotide sequences as set forth in SEQ ID NOS: 143, 145, 147, 149, 151, 153, and 155, or complementary oligonucleotides thereof.

The probe or probe set of the present invention specifically binds with PCR products amplified from target regions of antibiotic resistance genes expressed in antibiotic-resistant bacterial species by PCR using the primer set of the present invention. Thus, the probe or probe set of the present invention can discriminate antibiotic-resistant bacterial species. The probe or probe set of the present invention was designed by searching antibiotic-resistant bacterial species, in particular, bacterial species having resistance to aminoglycosides, beta-lactams, erythromycins, methicillins, penicillins, quinolones, and vancomycins, and genes related thereto, investigating the occurrence frequency of the genes in each country, and selecting genes having higher occurrence frequency as target sequences.

As used herein, the term “probe” refers to a single-stranded nucleic acid sequence that can be base-paired with a complementary single-stranded target sequence to form a double-stranded molecule (hybrid).

As used herein, the term “hybridization” refers to the bonding of two complementary strands of nucleic acid to form a double-stranded molecule (hybrid).

As used herein, “stringency” is the term used to describe a temperature and a solvent composition during hybridization and the subsequent processes. Under high stringency conditions, highly homologous nucleic acid hybrids will be formed. That is, hybrids with no sufficient degree of complementarity will not be formed. Accordingly, the stringency of the assay conditions determines the amount of complementarity which should exist between two nucleic acid strands to form a hybrid. Stringency is chosen to maximize the difference in stability between probe-target hybrids and probe-non-target hybrids.

The present invention also provides a microarray in which a substrate is immobilized with at least one oligonucleotide probe or probe set according to an embodiment of the present invention.

As used herein, the term “microarray” refers to a high-density array of groups of polynucleotides immobilized on a substrate. Here, each polynucleotide group is a microarray immobilized in predetermined regions of the substrate. The microarray is well known in the art. Examples of such microarrays are disclosed in U.S. Pat. Nos. 5,445,934 and 5,744,305, the disclosures of which are incorporated herein in their entireties by reference. The oligonucleotide probe and probe set are as described above.

The present invention also provides a method of detecting bacterial species having resistance to at least one selected from aminoglycoside-based, beta lactam-based, erythromycin-based, methicillin-based, vancomycin-based, and quinolone-based antibiotics, the method including:

contacting a sample with at least one oligonucleotide probe or probe set according to an embodiment of the present invention so that a target sequence of the sample hybridizes with a probe sequence; and

detecting degree of hybridization between the probe sequence and the target sequence of the sample.

The method of the present invention may further include, after detecting the degree of hybridization:

determining that bacterial species having resistance to an aminoglycoside-based antibiotic is present in the sample when it is determined that at least one gene selected from the group consisting of aataph, ant, and aph is present;

determining that bacterial species having resistance to a beta-lactam-based antibiotic is present in the sample when it is determined that at least one gene selected from the group consisting of CMY1, CMY2, CTX1, CTX2, DHA, IMP, OXA, PER, SHV, TEM, and VIM is present;

determining that bacterial species having resistance to an erythromycin-based antibiotic is present in the sample when it is determined that at least one gene selected from the group consisting of ermA, ermB, ermC, and mef is present;

determining that bacterial species having resistance to a methicillin-based antibiotic is present in the sample when it is determined that a mecA gene is present;

determining that bacterial species having resistance to a vancomycin-based antibiotic is present in the sample when it is determined that at least one gene selected from the group consisting of vanA and vanB is present; and

determining that bacterial species having resistance to a quinolone-based antibiotic is present in the sample when it is determined that at least one gene selected from the group consisting of Pae mutant-type gyrA, Sau mutant-type gyrA, Sau mutant-type parC, Sau mutant-type parE, and Spn mutant-type pbp2b is present. Here, “mutation” occurred in the mutant-type genes is as presented in probes as set forth in SEQ ID NOS: 106-155 (see Table 5 below).

The method of the present invention may further include, after detecting the degree of hybridization: determining that bacterial species having resistance to a quinolone-based antibiotic is absent in the sample when it is determined that at least one gene selected from the group consisting of Pae wild-type gyrA, Sau wild-type gyrA, Sau wild-type parC, Sau wild-type parE, and Spn wild-type pbp2b is present.

In the method of the present invention, the antibiotic-resistant bacterial species may include Spn, Sau, Kpn, Mca, Hin, Kpn, Eco, Pae, Mpn, Cpn, and Lpn.

In the method of the present invention, the sample may include a PCR product obtained by PCR using, as primers, a primer set according to an embodiment of the present invention, and, as templates, nucleic acids in the sample. The PCR may include both single PCR and multiplex PCR.

In the method of the present invention, the nucleic acid may be selected from the group consisting of chromosomal DNA, cDNA, and a fragment thereof.

In the method of the present invention, the target sequence may be labeled with a detectable labeling material. For example, the labeling material may be a fluorescent material, a phosphorescent material, or a radioactive material. Preferably, the labeling material may be Cy-5 or Cy-3.

In the method of the present invention, the probe or probe set may be immobilized on a microarray substrate.

In the method of the present invention, the hybridization between the target sequence and the probe sequence may be performed under a high stringency hybridization condition. For example, the high stringency hybridization condition may include a 0.12M phosphate buffer (65° C.) including equal moles of Na₂HPO₄ and NaH₂PO₄, 1 mM EDTA, and 0.02% sodium dodecylsulfate.

In the method of the present invention, the “PCR” refers to a polymerase chain reaction and is a method for amplifying a target nucleic acid from a primer pair specifically binding with the target nucleic acid using a polymerase. PCR is well known in the art. PCR can also be performed using a commercially available kit. PCR can be classified into single PCR for amplification of only a single target sequence in a single PCR reaction and into multiplex PCR for simultaneous amplification of different target sequences in a single PCR reaction. Multiplex PCR is performed using a plurality of primer pairs.

In the method of the present invention, the detection of at least one antibiotic-resistant bacterial species can be achieved by labeling a PCR product with a detectable signal-emitting material; hybridizing the labeled PCR product with the at least one oligonucleotide probe or probe set; and detecting a signal generated from the hybridization product. The detectable signal may be an optical signal or an electrical signal, but the present invention is not limited thereto. An optically active material may be a fluorescent material or a phosphorescent material. The fluorescent material may be fluorescein, Cy-5, or Cy-3. A PCR product may be unlabeled or labeled with a detectable signal-emitting material before or after hybridization. In a case where a PCR product is unlabeled, hybridization between the PCR product and a probe oligonucleotide can be detected by an electrical signal, but the present invention is not limited thereto.

The present invention also provides a kit for detecting bacterial species having resistance to at least one selected from the group consisting of aminoglycoside-based, beta-lactam-based, erythromycin-based, methicillin-based, vancomycin-based, and quinolone-based antibiotics in a sample, the kit including a primer set according to an embodiment of the present invention and an instruction manual.

In the kit of the present invention, the primer set is as described above. The instruction manual includes a description specified so that the primer set can be used as amplification primers for amplification of antibiotic resistance genes expressed in antibiotic-resistant bacterial species. When a product specific to an antibiotic resistance gene is obtained by an amplification reaction (e.g., PCR) using the kit including the primer set, it is determined that antibiotic-resistant bacterial species are present in the sample. The kit may include an amplification reagent and a detectable labeling material.

The kit of the present invention may further include an oligonucleotide probe or probe set according to an embodiment of the present invention. The probe or probe set can detect a product obtained by amplification reaction using the primer set as primers.

In the kit of the present invention, the antibiotic-resistant bacterial species may include Spn, Sau, Kpn, Mca, Hin, Kpn, Eco, Pae, Mpn, Cpn, and Lpn, but the present invention is not limited thereto.

Hereinafter, the present invention will be described more specifically with reference to the following examples. The following examples are only for illustrative purposes and are not intended to limit the scope of the invention.

### EXAMPLES

### Example 1

### Selection of Antibiotic-Resistant Bacterial Species, Antibiotic Resistance Genes Thereof, and Primers for Amplifying the Genes

In Example 1, antibiotic-resistant bacterial species, mainly respiratory disease-causing bacterial species and antibiotic resistance genes expressed in the bacterial species were selected, and primer sets capable of amplifying the genes and probes were designed.

(1) Design of Primers

First, respiratory disease-causing bacterial species and antibiotic resistance genes specific to the bacterial species were selected by searching respiratory disease-associated database (e.g., http://medinfo.ufl.edu/year2/mmid/bms5300/bugs/virufact.html, which is produced and maintained by University of Florida, Colledg of Medicine) and related documents. Aminoglycosides, beta-lactams, quinolones, erythromycins, methicillins, penicillins, and vancomycins were used as antibiotics.

Primers were designed from the antibiotic resistance genes of the selected respiratory disease-causing bacterial species. That is, primers specific to the antibiotic resistance genes were designed from the antibiotic resistance genes. In the primer design, thermodynamic coefficients for potential primer sequences were determined using parameters from Santalucia et al. [Santalucia J, Proc. Natl. Acad. Sci. USA 95:1460-1465 (1998)]. Variables for primer design were as follows: the number of ambiguous nucleotide: 0, GC content: 30-70%, non-specifically matched base pairs: <4 bp, <10 contiguous base pairs with other gene sequence, primer length: 19-24 bases, not contain repetitive nucleotides, Δ G=137078-162324, Δ Tm=10° C., amplicon length: 60-400 bp.

The process of selecting primers is as follows: Firstly, unique region for primer design was selected by the criteria, ambiguous nucleotide is 0, that is, there is no variant alleles, GC percent is in the range of 30-70%, elite pair was selected when there is no more than 12 bp contiguous sequence identical with sequences in other species. The length of primer is 19-24 bp. Secondly, the candidate primer pairs were selected by the criteria, amplicon length is 60-400 bp, a primer pair which satisfy minimum length of elite pair, 9 bp or less. Thirdly, the candidate primer pairs were ranked by the criteria, in the order from small to large length of the elite pair length and from lower to higher delta TM. Fourthly, the selected primer pairs were tested, and the selected primer pairs were removed form the candidate when they produce monomer in a PCR at 72° C. or more of polymerizaton temperature and at 62° C. annealing temperature or when they are searched by using Blastn and the search results show that e-value <0.05 with sequences in other species.

As a result, primer sets targeting the antibiotic resistance genes presented in Table 2 above were designed.

(2) Design of Probes

Probes were selected based on respective amplified regions of the antibiotic resistance genes using DNAstar program and are summarized in Table 5 below. Probes were selected from the region between the forward primer and reverse primer in the targe sequence. Firstly, unique region for probe design was selected from the region between the forward primer and reverse primer in the targe sequence, by the following criteria, ambiguous nucleotide is 0, that is, there is no variant alleles, GC percent is in the range of 30-70%, elite pair was selected when there is no more than 12 bp contiguous sequence identical with sequences in other species. The length of probe is 20-24 bp. Secondly, probes were selected from the selected unique sequence present in the region between the forward primer and reverse primer.

TABLE 5

| | | | | | |
|---|---|---|---|---|---|
| | | | | Binding | |
| Antibiotic | Gene | Type | Probe sequence | position | SEQ ID NO: |

| | | | | | |
|---|---|---|---|---|---|
| Aminoglycoside | aataph | | TAATTCATGTTCTGGCAAATCTTC | 469 | 53 |
| Aminoglycoside | aataph | | TAGTGGTTATGATAGTGTGGCATA | 627 | 54 |
| Aminoglycoside | aataph | | TAACAATCTTCTTTTTTGCCCTCG | 495 | 55 |
| Aminoglycoside | ant | | GTTATGACCATCTGTGCCAGTTCG | 620 | 56 |
| Aminoglycoside | ant | | CTACGATAAGGGCACAAATCGCA | 408 | 57 |
| Aminoglycoside | aph | | GAACTTGTCTTTTCCCACGGCGAC | 2010 | 58 |
| Aminoglycoside | aph | | GCTTTCCTTCCAGCCATAGCATCA | 1651 | 59 |
| Beta lactam | CMY1 | | CAATTCCCCGAGGAGGTGGATT | 430 | 60 |
| Beta lactam | CMY1 | | GTGGTCAAGGGAGCGATGCAG | 304 | 61 |
| Beta lactam | CMY2 | | ACCCTCAGGAATGAGTTACGAAGA | 552 | 62 |
| Beta lactam | CMY2 | | TCTTCGTAACTCATTCCTGAGGGT | 552 | 63 |
| Beta lactam | CMY2 | | GGCGGTGAAACCCTCAGGAATGAG | 543 | 64 |
| Beta lactam | CTX1 | | GGACGATGTCACTGGCTGAGC | 353 | 65 |
| Beta lactam | CTX1 | | GACGTGCTTTTCCGCAATCGGAT | 326 | 66 |
| Beta lactam | CTX2 | | GTATTCAGCGTAGGTTCAGTGCG | 499 | 67 |
| Beta lactam | CTX2 | | ATGGCGGTATTCAGCGTAGGTTC | 505 | 68 |
| Beta lactam | DHA | | ATTACTGTGCCGGAAAGTGCGCA | 724 | 69 |
| Beta lactam | DHA | | ATCATTAACGGTGTGACCAACGA | 1006 | 70 |
| Beta lactam | IMP | | TATTATTCGGTGGTTGTTTT | 497 | 71 |
| Beta lactam | IMP | | AACTGGTTGTTCCAAGTCAC | 611 | 72 |
| Beta lactam | IMP | | AAATATGGTAAGGCAAAACT | 595 | 73 |
| Beta lactam | OXA | | AGCCATGCTTCTGTTAATCCGTT | 549 | 74 |
| Beta lactam | OXA | | ACGCAGGAATTGAATTTGTTC | 591 | 75 |
| Beta lactam | PER | | GTAAACAGGGCTAAGGTTTT | 440 | 76 |
| Beta lactam | PER | | CAGAATACCTGGGCTCCGAT | 461 | 77 |
| Beta lactam | SHV | | GTGACGAACAGCTGGAGCGAA | 248 | 78 |
| Beta lactam | SHV | | GTGGATGCCGGTGACGAACAG | 238 | 79 |
| Beta lactam | TEM | | CTCGTCGTTTGGTATGGCTTCAT | 503 | 80 |
| Beta lactam | TEM | | TGGCTTCATTCAGCTCCGGTTC | 490 | 81 |
| Beta lactam | VIM | | CTGAGCGATTTGTGTGCGCTTTT | 799 | 82 |
| Beta lactam | VIM | | CTCAGTCGTTGAGTAGCAGGCA | 817 | 83 |
| Erythromycin | ermA | | ATTAATGGTGGAGATGGAT | 435 | 84 |
| Erythromycin | ermA | | TCTGCAACGAGCTTTGGGTTTAC | 411 | 85 |
| Erythromycin | ermB | | GTGGTTTTTGAAAGCCATGCG | 337 | 86 |
| Erythromycin | ermB | | TGCGTCTGACATCTATCTGAT | 354 | 87 |
| Erythromycin | ermC | | AGAGGGTTATAATGAACGAGAA | 130 | 88 |
| Erythromycin | ermC | | AAATACAAAACGCTCATTGGC | 548 | 89 |
| Erythromycin | ermC | | AAGAGGGTTATAATGAACGAGAAA | 129 | 90 |
| Erythromycin | ermC | | TTTGAAATCGGCTCAGGAAAA | 243 | 91 |
| Erythromycin | ermC | | ACAAAACGCTCATTGGCATTA | 552 | 92 |
| Erythromycin | mef | | TGTCTATGGCTTCATTAGTAGGTT | 142 | 93 |
| Erythromycin | mef | | CCATTTGCAGGATGGCACTAGTGA | 73 | 94 |
| Erythromycin | mef | | TGGCTTCATTAGTAGGTTTTTTAC | 148 | 95 |
| Methicillin | mecA | | TGCTTCTGCAGGATCTTGGTTTGG | 3169 | 96 |
| Methicillin | mecA | | CAAGTGCTAATAATTCACCTGTT | 1151 | 97 |
| Methicillin | mecA | | GTATGGCATGAGTAACGAAGA | 1208 | 98 |
| Methicillin | mecA | | AAATCAGAATCAAGAAGTGCTC | 2982 | 99 |
| Methicillin | mecA | | CAGTACCTGAGCCATAATCATT | 1116 | 100 |
| Methicillin | mecA | | TTTATGTATGGCATGAGTAACG | 1203 | 101 |
| Vancomycin | vanA | | CATTCCGCGCAAGGTTTTTCGCA | 154 | 102 |
| Vancomycin | vanA | | CGTTGACATACATCGTTGCGAA | 401 | 103 |
| Vancomycin | vanB | | ACGGCAAAGAAAGTATATCGGG | 1000 | 104 |
| Vancomycin | vanB | | CCTGATGGATGCGGAAGATACC | 892 | 105 |
| Quinolone | Pae gyrA | wp | aagaaatccGCCcgwgtggt | 454 | 106 |
| Quinolone | Pae gyrA | mp | aagaaatccTCCcgwgtggt | 454 | 107 |
| Quinolone | Pae gyrA | wp | aaatcckcycgTgtggtcggcg | 457 | 108 |
| Quinolone | Pae gyrA | mp | aaatcckcycgAgtggtcggcg | 457 | 109 |
| Quinolone | Pae gyrA | wp | tcgccgtgCgggtggt | 785 | 110 |
| Quinolone | Pae gyrA | mp | tcgccgtgTgggtggt | 785 | 111 |
| Quinolone | Pae gyrA | wp | cggcgacaCcscrgtcta | 504 | 112 |
| Quinolone | Pae gyrA | mp | cggcgacaTcscrgtcta | 504 | 113 |
| Quinolone | Pae gyrA | wp | cscrgtctacGacaccatcgt | 513 | 114 |
| Quinolone | Pae gyrA | mp | cscrgtctacCacaccatcgt | 513 | 115 |
| Quinolone | Pae gyrA | wp | cacgatggtGTCgtagacygsg | 513 | 116 |
| Quinolone | Pae gyrA | mp | cacgatggtTGGgtagacygsg | 513 | 117 |
| Quinolone | Pae gyrA | wp | cscrgtctacGacaccatcgt | 513 | 118 |
| Quinolone | Pae gyrA | mp | cscrgtctacAacaccatcgt | 513 | 119 |
| Quinolone | Pae gyrA | wp1 | cscrgtctacGacaccatcgt | 513 | 120 |
| Quinolone | Pae gyrA | mp1 | cscrgtctacAacaccatcgt | 513 | 121 |
| Quinolone | Pae gyrA | wp2 | cscrgtctacGacaccatcgtc | 513 | 122 |
| Quinolone | Pae gyrA | mp2 | cscrgtctacAacaccatcgtc | 513 | 123 |
| Quinolone | Sau gyrA | wp | ctcatggtgactCayctatytat | 239 | 124 |
| Quinolone | Sau gyrA | mp | ctcatggtgactTayctatytat | 239 | 125 |
| Quinolone | Sau gyrA | wp | catggtgactIaTCTatytatrIagc | 241 | 126 |
| Quinolone | Sau gyrA | mp | catggtgactIaCCTatytatrIagc | 241 | 127 |
| Quinolone | Sau gyrA | wp | tIayctatytatGAAgcaatggtac | 250 | 128 |
| Quinolone | Sau gyrA | mp | tIayctatytatAAAgcaatggtac | 250 | 129 |
| Quinolone | Sau gyrA | wp | cgtaccattgcTTCataratagrt | 252 | 130 |
| Quinolone | Sau gyrA | mp | cgtaccattgcTCCataratagrt | 252 | 131 |
| Quinolone | Sau parC | wp | acayggagactCctcrgtgtac | 228 | 132 |
| Quinolone | Sau parC | mp | acayggagactTctcrgtgtac | 228 | 133 |
| Quinolone | Sau parC | wp | acayggagactCctcrgtgtac | 228 | 134 |
| Quinolone | Sau parC | mp | acayggagactActcrgtgtac | 228 | 135 |
| Quinolone | Sau parC | wp | accattgcTTCgtacacygag | 252 | 136 |
| Quinolone | Sau parC | mp | accattgcTTTgtacacygag | 252 | 137 |
| Quinolone | Sau parE | wp | aaaaayacwgaAaaaaatgaattg | 1255 | 138 |
| Quinolone | Sau parE | mp | aaaaayacwgaTaaaaatgaattg | 1255 | 139 |
| Quinolone | Sau parE | wp | ccgattgtgtGgataattgtat | 1421 | 140 |
| Quinolone | Sau parE | mp | ccgattgtgtAgataattgtat | 1421 | 141 |
| Penicillin | Spn pbp2b | wp | tattcatcHaatACCtayatggtIca | 721 | 142 |
| Penicillin | Spn pbp2b | mp | tattcatcHaatGCTtayatggtIca | 721 | 143 |
| Penicillin | Spn pbp2b | wp | attcatcwaatACCtayatggtIca | 814 | 144 |
| Penicillin | Spn pbp2b | mp | attcatcwaatGCTtayatggtIca | 814 | 145 |
| Penicillin | Spn pbp2b | wp | cIgctatggAGaaaytkcgtIc | 853 | 146 |
| Penicillin | Spn pbp2b | mp | cIgctatggGAaaaytkcgtIc | 853 | 147 |
| Penicillin | Spn pbp2b | wp | gcttgggbActgcgac | 853 | 148 |
| Penicillin | Spn pbp2b | mp | gcttgggbGctgcgac | 853 | 149 |
| Penicillin | Spn pbp2b | wp | gcttgggbActgcgachg | 853 | 150 |
| Penicillin | Spn pbp2b | mp | gcttgggbGctgcgachg | 853 | 151 |
| Penicillin | Spn pbp2b | wp | gYttgggbActgcgac | 853 | 152 |
| Penicillin | Spn pbp2b | mp | gYttgggbTctgcgac | 853 | 153 |
| Penicillin | Spn pbp2b | wp | tggYttgIgbActgcgacIgg | 851 | 154 |
| Penicillin | Spn pbp2b | mp | tggYttgIgbTctgcgacIgg | 851 | 155 |

In Table 5, wp and mp represent wild-type and mutant-type probes, respectively, Spn represents Streptococcus pneumoniae, Pae represents Pseudomonas aeruginosa, Sau represents Staphylococcus aureus, and I represents inosine.

### Example 2

### Amplification of Antibiotic Resistance Genes Expressed in Antibiotic-Resistant Bacterial Species Using Primer Sets of the Present Invention

The antibiotic resistance genes expressed in the antibiotic-resistant bacterial species presented in Table 2 above were amplified by single PCR and multiplex PCR using the primer sets designed in Example 1. 5′-ends of all the forward and reverse primers were labeled with Cy-3. Oligonucleotides as set forth in SEQ ID NOS: 1-52 (26 primer sets) were used as primers.

(1) Preparation of Bacterial Cultures

Cultural isolates of 11 antibiotic-resistant bacterial species provided from Asian-Pacific Research Foundation for Infectious Diseases (ARFID) were used. The 11 antibiotic-resistant bacterial species were Spn, Sau, Kpn, Mca, Hin, Kpn, Eco, Pae, Mpn, Cpn, and Lpn.

(2) Single PCR

First, single PCR was performed using each of 21 primer sets (SEQ ID NOS: 1 and 2 for aataph, SEQ ID NOS: 3 and 4 for ant, SEQ ID NOS: 5 and 6 for aph, SEQ ID NOS: 7 and 8 for CMY1, SEQ ID NOS: 9 and 10 for CMY2, SEQ ID NOS: 11 and 12 for CTX1, SEQ ID NOS: 13 and 14 for CTX2, SEQ ID NOS: 15 and 16 for DHA, SEQ ID NOS: 17 and 18 for IMP, SEQ ID NOS: 19 and 20 for OXA, SEQ ID NOS: 21 and 22 for PER, SEQ ID NOS: 23 and 24 for SHV, SEQ ID NOS: 25 and 26 for TEM, SEQ ID NOS: 27 and 28 for VIM, SEQ ID NOS: 29 and 30 for ermA, SEQ ID NOS: 31 and 32 for ermB, SEQ ID NOS: 33 and 34 for ermC, SEQ ID NOS: 35 and 36 for mef, SEQ ID NOS: 37 and 38 for mecA, SEQ ID NOS: 49 and 50 for vanA, and SEQ ID NOS: 51 and 52 for vanB), and as templates, genomic DNAs corresponding to each primer set.

Also, single PCR was performed using each of five primer sets (SEQ ID NOS: 39 and 40 for Spn pbp2b, SEQ ID NOS: 41 and 42 for Pae gyrA, SEQ ID NOS: 43 and 44 for Sau gyrA, SEQ ID NOS: 45 and 46 for Sau parC, and SEQ ID NOS: 47 and 48 for Sau parE), and as templates, genomic DNAs corresponding to each primer set.

The single PCR was performed using 20 μl of a PCR solution of 2 μl of a genomic DNA (extracted using a G-spin genomic DNA extraction kit, iNtRON) in a mixed solution including 1.5 mM of MgCl₂, 250 mM of each dNTP, 10 mM tris-HCl (pH 9.0), 1 unit of Taq polymerase, and about 2 pmol of each primer, for 29 minutes and 5 seconds, as follows: 25 cycles of denaturation at 95° C. for 10 seconds, annealing at 60° C. for 10 seconds, and extension at 60° C. for 13 seconds.

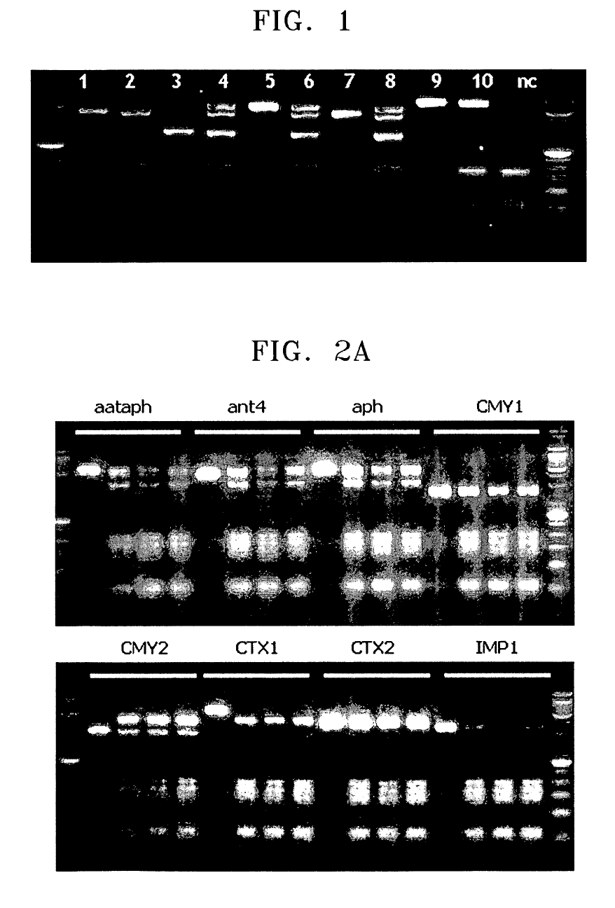

As a result, target sequences of the antibiotic resistance genes of the 11 antibiotic-resistant bacterial species were specifically amplified by the single PCR. FIG. 1 shows the results of the single PCR performed using each of the five primer sets (SEQ ID NOS: 39 and 40 for Spn pbp2b, SEQ ID NOS: 41 and 42 for Pae gyrA, SEQ ID NOS: 43 and 44 for Sau gyrA, SEQ ID NOS: 45 and 46 for Sau parC, and SEQ ID NOS: 47 and 48 for Sau parE), and as templates, the genomic DNAs corresponding to each primer set, and the results of multiplex PCR performed using all the five primer sets, and as templates, genomic DNAs of each bacterial species. In FIG. 1, lane 1 shows the results of single PCR performed using the primer set for Spn pbp2b (SEQ ID NOS: 39 and 40), and as templates, genomic DNAs of Spn, lane 3 shows the results of single PCR performed using the primer set for Pae gyrA (SEQ ID NOS: 41 and 42), and as templates, genomic DNAs of Pae, lane 5 shows the results of single PCR performed using the primer set for Sau gyrA (SEQ ID NOS: 43 and 44), and as templates, genomic DNAs of Sau, lane 7 shows the results of single PCR performed using the primer set for Sau parC (SEQ ID NOS: 45 and 46), and as templates, genomic DNAs of Sau, and lane 9 shows the results of single PCR performed using the primer set for Sau parE (SEQ ID NOS: 47 and 48), and as templates, genomic DNAs of Sau. Also, lanes 2, 4, 6, 8, and 10 show the results of multiplex PCR performed using all of the primer set for Spn pbp2b (SEQ ID NOS: 39 and 40), the primer set for Pae gyrA (SEQ ID NOS: 41 and 42), the primer set for Sau gyrA (SEQ ID NOS: 43 and 44), the primer set for Sau parC (SEQ ID NOS: 45 and 46), and the primer set for Sau parE (SEQ ID NOS: 47 and 48), and as templates, genomic DNAs of Spn, Pae, Sau, Sau, and Sau, respectively.

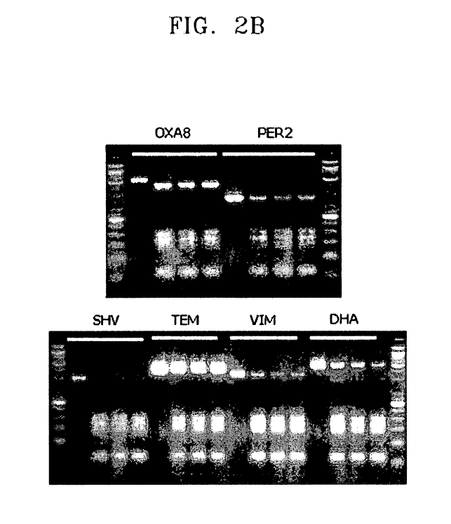

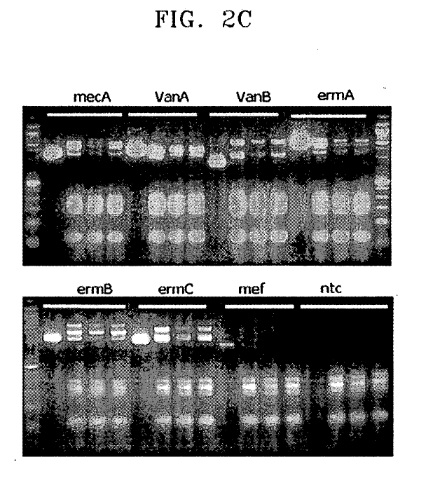

FIGS. 2A, 2B, and 2C show the results of single PCR performed using each of the 21 primer sets (i.e., SEQ ID NOS: 1 and 2 for aataph, SEQ ID NOS: 3 and 4 for ant, SEQ ID NOS: 5 and 6 for aph, SEQ ID NOS: 7 and 8 for CMY1, SEQ ID NOS: 9 and 10 for CMY2, SEQ ID NOS: 11 and 12 for CTX1, SEQ ID NOS: 13 and 14 for CTX2, SEQ ID NOS: 15 and 16 for DHA, SEQ ID NOS: 17 and 18 for IMP, SEQ ID NOS: 19 and 20 for OXA, SEQ ID NOS: 21 and 22 for PER, SEQ ID NOS: 23 and 24 for SHV, SEQ ID NOS: 25 and 26 for TEM, SEQ ID NOS: 27 and 28 for VIM, SEQ ID NOS: 29 and 30 for ermA, SEQ ID NOS: 31 and 32 for ermB, SEQ ID NOS: 33 and 34 for ermC, SEQ ID NOS: 35 and 36 for mef, SEQ ID NOS: 37 and 38 for mecA, SEQ ID NOS: 49 and 50 for vanA, and SEQ ID NOS: 51 and 52 for vanB), and as templates, genomic DNAs of each bacterial species containing at least one antibiotic resistance gene, and the results of multiplex PCR performed using all the 21 primer sets, and as templates, genomic DNAs of each bacterial species containing at least one antibiotic resistance gene. In lane groups of FIGS. 2A, 2B, and 2C, i.e., aataph, ant4, aph, CMY1, CMY2, CTX1, CTX2, IMP1, OXA8, PER2, SHV, TEM, VIM, DHA, mecA, VanA, VanB, ermA, ermB, ermC, and mef, DNAs of bacterial species (hereinafter, referred to as “target bacterial species”) in which antibiotic resistance genes presented in Table 6 below were inserted into plasmids were used as templates.

TABLE 6

| | | Genes of target bacterial | |
|---|---|---|---|
| Lane group | Target bacterial species | species | Remark |
| aataph | Sau | aataph, ant, aph | |
| ant4 | Sau | aataph, ant, aph | |
| aph | Sau | aataph, ant, aph | |
| CMY1 | Kpn | CMY1 | |
| CMY2 | Eco | TEM, CMY2 | |
| CTX1 | Kpn | SHV, CTX-1, OXA, TEM | |
| CTX2 | Eco | TEM, CTX-2 | |
| IMP1 | Acinetobacter genospecies 3 | IMP | A kind of Aba |
| OXA8 | Eco | OXA, TEM, CTX-1 | |
| PER2 | Aba | PER | |
| SHV | Kpn | SHV, OXA | |
| TEM | Enterobacter cloacae | DHA, TEM | |
| VIM | A. phenon 6/ct13TU | VIM | A kind of Aba |
| DHA | Enterobacter aerogenes | DHA, SHV | |
| mecA | Sau | aataph, ant, ermA, mecA | |
| VanA | Enterococcus faecalis | VanA | |
| VanB | Enterococcus faecalis | VanB | |
| ermA | Sau | aataph, ant, ermA, mecA | |
| ermB | Enterococcus faecalis | vanA, aataph, ermB, aph | |
| ermC | Sau | aataph, ant, ermC, mecA, mecA | |
| mef | S. pyogens mef | mef | |

In Table 6 above, some target bacterial species are not naturally occurring antibiotic-resistant bacterial species but are antibiotic-resistant bacterial transformants in which an antibiotic resistance gene-containing plasmid is introduced. As for some antibiotic-resistant bacterial species, naturally occurring bacterial species are not easily available due to a low case frequency, or are fatally risky, and thus, their bacterial transformants are used as a model.

In each lane group, lane 1: single PCR, lane 2: multiplex PCR; lanes 3 and 4: multiplex PCR in the presence of 0.5% betaine and 0.25% betaine, respectively.

As shown in FIGS. 1 and 2, in each single PCR, the target sequences were specifically amplified.

(3) Multiplex PCR

Multiplex PCR was performed using 21 primer sets (SEQ ID NOS: 1 and 2 for aataph, SEQ ID NOS: 3 and 4 for ant, SEQ ID NOS: 5 and 6 for aph, SEQ ID NOS: 7 and 8 for CMY1, SEQ ID NOS: 9 and 10 for CMY2, SEQ ID NOS: 11 and 12 for CTX1, SEQ ID NOS: 13 and 14 for CTX2, SEQ ID NOS: 15 and 16 for DHA, SEQ ID NOS: 17 and 18 for IMP, SEQ ID NOS: 19 and 20 for OXA, SEQ ID NOS: 21 and 22 for PER, SEQ ID NOS: 23 and 24 for SHV, SEQ ID NOS: 25 and 26 for TEM, SEQ ID NOS: 27 and 28 for VIM, SEQ ID NOS: 29 and 30 for ermA, SEQ ID NOS: 31 and 32 for ermB, SEQ ID NOS: 33 and 34 for ermC, SEQ ID NOS: 35 and 36 for mef, SEQ ID NOS: 37 and 38 for mecA, SEQ ID NOS: 49 and 50 for vanA, and SEQ ID NOS: 51 and 52 for vanB), and genomic DNAs of each bacterial species containing target gene(s).

Also, multiplex PCR was performed using five primer sets (SEQ ID NOS: 39 and 40 for Spn pbp2b, SEQ ID NOS: 41 and 42 for Pae gyrA, SEQ ID NOS: 43 and 44 for Sau gyrA, SEQ ID NOS: 45 and 46 for Sau parC, SEQ ID NOS: 47 and 48 for Sau parE), and genomic DNAs of each bacterial species containing target gene(s).

The PCR mix for the multiplex PCR was made up to a total volume of 50 μl, containing 10.5 μl of distilled water, 7.5 μl of 10× buffer (100 mM Tris-HCl, 500 mM KCl, 15 mM MgCl2, 0.1% Gelatine), 1 μl of 200 μM dNTP (each), 20 μl of 400 nM end-labeled primer (each, Bioneer, Korea), 5 μl of extracted genomic DNA, and 1 μl of Taq polymerase (5 units).

The multiplex PCR was performed as follows: initial denaturation at 95° C. for one minute; 25 cycles of denaturation at 95° C. for 5 seconds, annealing at 62° C. for 13 seconds, and extension at 72° C. for 15 seconds; and extension at 72° C. for one minute.

FIGS. 1 and 2(A, B, and C) are agarose gel electrophoretic results of PCR products obtained by multiplex PCR using 5 and 21 target sequences, respectively.

In Example 2, multiplex PCR products were hybridized with oligonucleotide probes (specific to the antibiotic resistance genes presented in Table 4 above) immobilized on microarrays, and fluorescence emitted from the microarrays were measured.

The probe-immobilized microarrays were manufactured as follows. First, wafers were spin-coated with a solution of GAPTES (γ-aminopropyltriethoxysilane) (20% (v/v)) or GAPDES (γ-aminopropyldiethoxysilane) (20% (v/v)) in ethanol. The spin coating was performed using a spin coater (Model CEE 70, CEE) as follows: initial coating at a rate of 500 rpm/10 sec and main coating at a rate of 2000 rpm/10 sec. After the spin coating was completed, the wafers were placed in a Teflon wafer carrier and cured at 120° C. for 40 minutes. The cured wafers were immersed in water for 10 minutes, ultrasonically washed for 15 minutes, immersed in water for 10 minutes, and dried. The drying was performed using a spin-drier. All the experiments were conducted in a clean room class 1000 where most dust particles had been sufficiently removed.

Oligonucleotide probe sets specific to the antibiotic resistance genes presented in Table 4 above were immobilized on the amino-activated wafers using a spotting method to thereby obtain microarrays.

The PCR products were added on the microarrays. The microarrays were incubated at 42° C. for one hour so that probe-target hybridization occurred and then washed with a washing buffer. Fluorescence intensity was measured using a GenePix Scanner (Molecular Device, U.S.A.).

An array of the probes spotted on the microarrays is presented in Table 7 below.

TABLE 7
microarray layout for determining antibiotic resistance of
bacterial species by detecting the presenece of target gene

| | Column 1-3 | Column 4-6 | Column 7-9 | Column 10-12 |
|---|---|---|---|---|
| Row 1 | 53 | 54 | 55 | 57 |
| Row 2 | 56 | 59 | 58 | 61 |
| Row 3 | 60 | 63 | 62 | 64 |
| Row 4 | 65 | 66 | 67 | 68 |
| Row 5 | 71 | 72 | 73 | 75 |
| Row 6 | 74 | 77 | 76 | 79 |
| Row 7 | 78 | 81 | 80 | 83 |
| Row 8 | 82 | 70 | 69 | 99 |
| Row 9 | 96 | 103 | 102 | 105 |
| Row 10 | 104 | 85 | 84 | 87 |
| Row 11 | 86 | 90 | 88 | 91 |
| Row 12 | 93 | 95 | 94 | + |
| Row 13 | + | + | 89 | 92 |
| Row 14 | 100 | 97 | 101 | 98 |

In Table 7, numbers represent the sequence identification numbers (SEQ ID NO) of the probes, and “+” represents a positive control probe.

TABLE 8
microarray layout for determining antibiotic resistance of
bacterial species by detecting the presence of mutation

| | Column 1-3 | Column 4-6 | Column 7-9 | Column 10-12 |
|---|---|---|---|---|
| Row 1 | 106 | 107 | 108 | 109 |
| Row 2 | 110 | 111 | 112 | 113 |
| Row 3 | 114 | 115 | 116 | 117 |
| Row 4 | 118 | 119 | 124 | 125 |
| Row 5 | 126 | 127 | 128 | 129 |
| Row 6 | 130 | 131 | 132 | 133 |
| Row 7 | 134 | 135 | 136 | 137 |
| Row 8 | 138 | 139 | 140 | 141 |
| Row 9 | 142 | 143 | 150 | 151 |
| Row 10 | 144 | 145 | 138 | 139 |
| Row 11 | 144 | 145 | + | + |
| Row 12 | 134 | 135 | 148 | 149 |
| Row 13 | 120 | 121 | 122 | 123 |
| Row 14 | 148 | 149 | − | − |

In Table 8, numbers represent the sequence identification numbers (SEQ ID NO) of the probes, and “+” and “−” represent a positive control probe and a negative control probe, respectively.

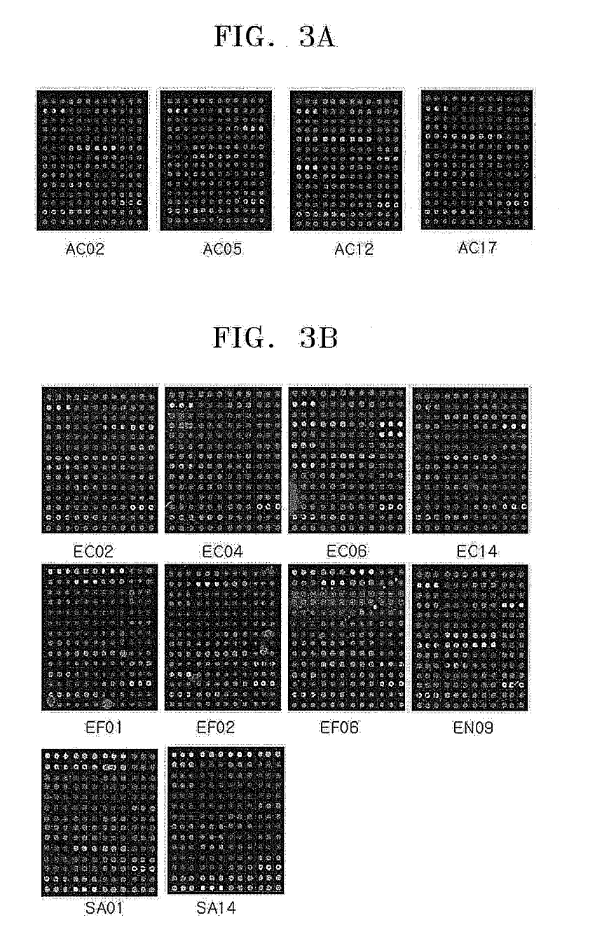

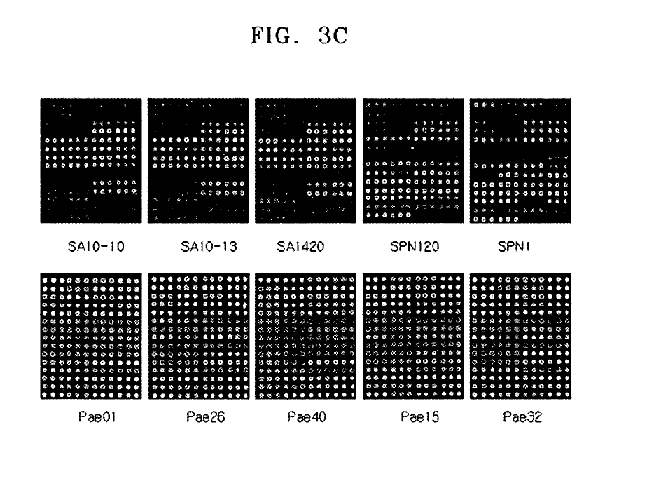

FIGS. 3A and 3B are images showing hybridization results of PCR products obtained by PCR using, as primers, all of the above-described 21 primer sets, and, as templates, genomic DNAs of predetermined antibiotic-resistant bacterial species, on a microarray having a specific oligonucleotide probe layout as presented in Table 7.

In FIGS. 3A and 3B, test bacterial species used for antibiotic resistance analysis and their antibiotic resistance genotypes are presented in Table 9 below. The antibiotic resistance genotypes were determined by PCR. As shown in FIGS. 3A and 3B, it can be determined whether or not bacterial species in a sample contains an antibiotic resistance gene by hybridization of multiple PCR products with probes immobilized on a microarray. Most antibiotic-resistant bacterial species had two or more antibiotic resistance genes.

TABLE 9
test bacterial species and antibiotic resistance genotypes

| Microarray | Bacterial species | Antibiotic resistance genotype(s) |
|---|---|---|
| AC02 | Aba | PER |
| AC05 | Aba | TEM |
| AC12 | Aba | VIM, IMP |
| AC17 | Aba | IMP |
| EC02 | Eco | SHV, TEM |
| EC04 | Eco | SHV, CTX-2 |
| EC06 | Eco | TEM, CTX-1, OXA |
| EC14 | Eco | TEM, CMY-2 |
| F01 | Enetrobacter faecalis | vanA, ermB, aac(6′)/aph(2″) |
| F02 | Enetrobacter faecalis | vanA, aph, ermB |
| F06 | Enetrobacter faecalis | vanA, aataph, aph, ermA, ermB |
| EN09 | Enterobacter facium | DHA, TEM |
| SA01 | Sau | aataph, aph, ermA, mecA |
| SA14 | Sau | aataph, ant4, ermC |

FIG. 3C is an image showing hybridization results of PCR products obtained by PCR using, as primers, all of the above-described five primer sets, and, as templates, genomic DNAs of predetermined antibiotic-resistant bacterial species, on a microarray having a specific oligonucleotide probe layout as presented in Table 8. As shown in FIG. 3C, it can be determined whether or not bacterial species in a sample contains an antibiotic resistance gene by hybridization of multiple PCR products with probes immobilized on a microarray. Here, the probes include probes specific to antibiotic resistance genes activated by mutation. In FIG. 3C, SA10-10, SA10-13, SA1420, SPN120, and Pae 01 represent serial numbers of samples.

A nucleic acid primer set according to the present invention can amplify antibiotic resistance gene(s) from antibiotic-resistant bacterial species.

A probe or probe set according to the present invention is specifically bound to a target sequence of a PCR product amplified using the primer set of the present invention, and thus, can be used to detect at least one antibiotic-resistant bacterial species.

A microarray according to the present invention can be used to detect at least one antibiotic-resistant bacterial species.

A detection method according to the present invention can efficiently detect antibiotic-resistant bacterial species with high specificity.

The terminology used herein is for the purpose of describing particular embodiments only and is not intended to be limiting of the invention. The terms “a” and “an” do not denote a limitation of quantity, but rather denote the presence of at least one of the referenced item. The term “or” means “and/or”. The terms “comprising”, “having”, “including”, and “containing” are to be construed as open-ended terms (i.e., meaning “including, but not limited to”).

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art to which this invention belongs.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. An oligonucleotide primer set comprising:
an oligonucleotide set comprising an oligonucleotide consisting of SEQ ID NO: 1 and an oligonucleotide consisting of SEQ ID NO: 2;
an oligonucleotide set comprising an oligonucleotide consisting of SEQ ID NO: 3 and an oligonucleotide consisting of SEQ ID NO: 4;
an oligonucleotide set comprising an oligonucleotide consisting of SEQ ID NO: 5 and an oligonucleotide consisting of SEQ ID NO: 6;
an oligonucleotide set comprising an oligonucleotide consisting of SEQ ID NO: 7 and an oligonucleotide consisting of SEQ ID NO: 8;
an oligonucleotide set comprising an oligonucleotide consisting of SEQ ID NO: 9 and an oligonucleotide consisting of SEQ ID NO: 10;
an oligonucleotide set comprising an oligonucleotide consisting of SEQ ID NO: 11 and an oligonucleotide consisting of SEQ ID NO: 12;
an oligonucleotide set comprising an oligonucleotide consisting of SEQ ID NO: 13 and an oligonucleotide consisting of SEQ ID NO: 14;
an oligonucleotide set comprising an oligonucleotide consisting of SEQ ID NO: 15 and an oligonucleotide consisting of SEQ ID NO: 16;
an oligonucleotide set comprising an oligonucleotide consisting of SEQ ID NO: 17 and an oligonucleotide consisting of SEQ ID NO: 18;
an oligonucleotide set comprising an oligonucleotide consisting of SEQ ID NO: 19 and an oligonucleotide consisting of SEQ ID NO: 20;
an oligonucleotide set comprising an oligonucleotide consisting of SEQ ID NO: 21 and an oligonucleotide consisting of SEQ ID NO: 22;
an oligonucleotide set comprising an oligonucleotide consisting of SEQ ID NO: 23 and an oligonucleotide consisting of SEQ ID NO: 24;
an oligonucleotide set comprising an oligonucleotide consisting of SEQ ID NO: 25 and an oligonucleotide consisting of SEQ ID NO: 26;
an oligonucleotide set comprising an oligonucleotide consisting of SEQ ID NO: 27 and an oligonucleotide consisting of SEQ ID NO: 28;
an oligonucleotide set comprising an oligonucleotide consisting of SEQ ID NO: 29 and an oligonucleotide consisting of SEQ ID NO: 30;
an oligonucleotide set comprising an oligonucleotide consisting of SEQ ID NO: 31 and an oligonucleotide consisting of SEQ ID NO: 32;
an oligonucleotide set comprising an oligonucleotide consisting of SEQ ID NO: 33 and an oligonucleotide consisting of SEQ ID NO: 34;
an oligonucleotide set comprising an oligonucleotide consisting of SEQ ID NO: 35 and an oligonucleotide consisting of SEQ ID NO: 36;
an oligonucleotide set comprising an oligonucleotide consisting of SEQ ID NO: 37 and an oligonucleotide consisting of SEQ ID NO: 38;
an oligonucleotide set comprising an oligonucleotide consisting of SEQ ID NO: 49 and an oligonucleotide consisting of SEQ ID NO: 50; and
an oligonucleotide set comprising an oligonucleotide consisting of SEQ ID NO: 51 and an oligonucleotide consisting of SEQ ID NO: 52;
wherein the oligonucleotide primer set specifically amplifies a target sequence selected from the group consisting of aataph, ant, aph, CMY1, CMY2, CTX1, CTX2, DHA, IMP, OXA, PER, SHV, TEM, VIM, ermA, ermB, ermC, mef, mecA, vanA, and vanB genes.

2. The oligonucleotide primer set of claim 1, further comprising:
an oligonucleotide set comprising an oligonucleotide consisting of SEQ ID NO: 39 and an oligonucleotide consisting of SEQ ID NO: 40;
an oligonucleotide set comprising an oligonucleotide consisting of SEQ ID NO: 41 and an oligonucleotide consisting of SEQ ID NO: 42;
an oligonucleotide set comprising an oligonucleotide consisting of SEQ ID NO: 43 and an oligonucleotide consisting of SEQ ID NO: 44;
an oligonucleotide set comprising an oligonucleotide consisting of SEQ ID NO: 45 and an oligonucleotide consisting of SEQ ID NO: 46; and
an oligonucleotide set comprising an oligonucleotide consisting of SEQ ID NO: 47 and an oligonucleotide consisting of SEQ ID NO: 48.

3. A microarray comprising a substrate and the oligonucleotide probe set immobilized thereon,
wherein the oligonucleotide probe set comprising:
an oligonucleotide set comprising oligonucleotides of SEQ ID NOS: 53-55 or a complement thereof, wherein the oligonucleotide can specifically hybridize with a nucleotide region from position 425 to position 890 of the aataph gene and does not cross-hybridize with any of the following genes: ant, aph, CMY1, CMY2, CTX1, CTX2, DHA, IMP, OXA, PER, SHV, TEM, VIM, ermA, ermB, ermC, mef, mecA, Spn pbp2b, Pae gyrA, Sau gyrA, Sau parC, Sau parE, vanA, and vanB;
an oligonucleotide set comprising oligonucleotides of SEQ ID NOS: 56-57 or a complement thereof, wherein the oligonucleotide specifically hybridizes with a nucleotide region from position 343 to position 722 of the ant gene and does not cross-hybridize with any of the following genes: aataph, aph, CMY1, CMY2, CTX1, CTX2, DHA, IMP, OXA, PER, SHV, TEM, VIM, ermA, ermB, ermC, mef, mecA, Spn pbp2b, Pae gyrA, Sau gyrA, Sau parC, Sau parE, vanA, and vanB;
an oligonucleotide set comprising an oligonucleotide of SEQ ID NOS: 58-59 or a complement thereof, wherein the oligonucleotide specifically hybridizes with a nucleotide region from position 1618 to position 2081 of the aph gene and does not cross-hybridize with any of the following genes: aataph, ant, CMY1, CMY2, CTX1, CTX2, DHA, IMP, OXA, PER, SHV, TEM, VIM, ermA, ermB, ermC, mef, mecA, Spn pbp2b, Pae gyrA, Sau gyrA, Sau parC, Sau parE, vanA, and vanB;
an oligonucleotide set comprising an oligonucleotide of SEQ ID NOS: 60 to 61 or a complement thereof, wherein the oligonucleotide specifically hybridizes with a nucleotide region from position 256 to position 449 of the CMY1 gene and does not cross-hybridize with any of the following genes: aataph, ant, aph, CMY2, CTX1, CTX2, DHA, IMP, OXA, PER, SHV, TEM, VIM, ermA, ermB, ermC, mef, mecA, Spn pbp2b, Pae gyrA, Sau gyrA, Sau parC, Sau parE, vanA, and vanB;
an oligonucleotide set comprising an oligonucleotide of SEQ ID NOS: 62-64 or a complement thereof, wherein the oligonucleotide specifically hybridizes with a nucleotide region from position 508 to position 738 of the CMY2 gene and does not cross-hybridize with any of the following genes:
aataph, ant, aph, CMY1, CTX1, CTX2, DHA, IMP, OXA, PER, SHV, TEM, VIM, ermA, ermB, ermC, mef, mecA, Spn pbp2b, Pae gyrA, Sau gyrA, Sau parC, Sau parE, vanA, and vanB;
an oligonucleotide set comprising an oligonucleotide of SEQ ID NOS: 65-66 or a complement thereof, wherein the oligonucleotide specifically hybridizes with a nucleotide region from position 55 to position 571 of the CTX1 gene and does not cross-hybridize with any of the following genes: aataph, ant, aph, CMY1, CMY2, CTX2, DHA, IMP, OXA, PER, SHV, TEM, VIM, ermA, ermB, ermC, mef, mecA, Spn pbp2b, Pae gyrA, Sau gyrA, Sau parC, Sau parE, vanA, and vanB;
an oligonucleotide set comprising an oligonucleotide of SEQ ID NOS: 67-68 or a complement thereof, wherein the oligonucleotide specifically hybridizes with a nucleotide region from position 346 to position 688 of the CTX2 gene and does not cross-hybridize with any of the following genes: aataph, ant, aph, CMY1, CMY2, CTX1, DHA, IMP, OXA, PER, SHV, TEM, VIM, ermA, ermB, ermC, mef, mecA, Spn pbp2b, Pae gyrA, Sau gyrA, Sau parC, Sau parE, vanA, and vanB;
an oligonucleotide set comprising an oligonucleotide of SEQ ID NOS: 69-70 or a complement thereof, wherein the oligonucleotide specifically hybridizes with a nucleotide region from position 630 to position 1045 of the DHA gene and does not cross-hybridize with any of the following genes: aataph, ant, aph, CMY1, CMY2, CTX1, CTX2, IMP, OXA, PER, SHV, TEM, VIM, ermA, ermB, ermC, mef, mecA, Spn pbp2b, Pae gyrA, Sau gyrA, Sau parC, Sau parE, vanA, and vanB;
an oligonucleotide set comprising an oligonucleotide of SEQ ID NOS: 71-73 or a complement thereof, wherein the oligonucleotide specifically hybridizes with a nucleotide region from position 361 to position 639 of the IMP gene and does not cross-hybridize with any of the following genes: aataph, ant, aph, CMY1, CMY2, CTX1, CTX2, DHA, OXA, PER, SHV, TEM, VIM, ermA, ermB, ermC, mef, mecA, Spn pbp2b, Pae gyrA, Sau gyrA, Sau parC, Sau parE, vanA, and vanB;
an oligonucleotide set comprising an oligonucleotide of SEQ ID NOS: 74-75 or a complement thereof, wherein the oligonucleotide specifically hybridizes with a nucleotide region from position 436 to position 865 of the OXA gene and does not cross-hybridize with any of the following genes: aataph, ant, aph, CMY1, CMY2, CTX1, CTX2, DHA, IMP, PER, SHV, TEM, VIM, ermA, ermB, ermC, mef, mecA, Spn pbp2b, Pae gyrA, Sau gyrA, Sau parC, Sau parE, vanA, and vanB;
an oligonucleotide set comprising an at oligonucleotide of SEQ ID NOS: 76-77 or a complement thereof, wherein the oligonucleotide specifically hybridizes with a nucleotide region from position 370 to position 559 of the PER gene and does not cross-hybridize with any of the following genes: aataph, ant, aph, CMY1, CMY2, CTX1, CTX2, DHA, IMP, OXA, SHV, TEM, VIM, ermA, ermB, ermC, mef, mecA, Spn pbp2b, Pae gyrA, Sau gyrA, Sau parC, Sau parE, vanA, and vanB;
an oligonucleotide set comprising an oligonucleotide of SEQ ID NOS: 78-79 or a complement thereof, wherein the oligonucleotide specifically hybridizes with a nucleotide region from position 116 to position 336 of the SHV gene and does not cross-hybridize with any of the following genes: aataph, ant, aph, CMY1, CMY2, CTX1, CTX2, DHA, IMP, OXA, PER, TEM, VIM, ermA, ermB, ermC, mef, mecA, Spn pbp2b, Pae gyrA, Sau gyrA, Sau parC, Sau parE, vanA, and vanB;
an oligonucleotide set comprising an oligonucleotide of SEQ ID NOS: 80-81 or a complement thereof, wherein the oligonucleotide specifically hybridizes with a nucleotide region from position 425 to position 783 of the TEM gene and does not cross-hybridize with any of the following genes: aataph, ant, aph, CMY1, CMY2, CTX1, CTX2, DHA, IMP, OXA, PER, SHV, VIM, ermA, ermB, ermC, mef, mecA, Spn pbp2b, Pae gyrA, Sau gyrA, Sau parC, Sau parE, vanA, and vanB;
an oligonucleotide set comprising an oligonucleotide of SEQ ID NOS: 82-83 or a complement thereof, wherein the oligonucleotide specifically hybridizes with a nucleotide region from position 572 to 848 of the VIM gene and does not cross-hybridize with any of the following genes: aataph, ant, aph, CMY1, CMY2, CTX1, CTX2, DHA, IMP, OXA, PER, SHV, TEM, ermA, ermB, ermC, mef, mecA, Spn pbp2b, Pae gyrA, Sau gyrA, Sau parC, Sau parE, vanA, and vanB;
an oligonucleotide set comprising an oligonucleotide of SEQ ID NOS: 84-85 or a complement thereof, wherein the oligonucleotide specifically hybridizes with a nucleotide region from position 138 to position 597 of the ermA gene and does not cross-hybridize with any of the following genes: aataph, ant, aph, CMY1, CMY2, CTX1, CTX2, DHA, IMP, OXA, PER, SHV, TEM, VIM, ermB, ermC, mef, mecA, Spn pbp2b, Pae gyrA, Sau gyrA, Sau parC, Sau parE, vanA, and vanB;
an oligonucleotide set comprising an oligonucleotide of SEQ ID NOS: 86-87 or a complement thereof, wherein the oligonucleotide specifically hybridizes with a nucleotide region from position 127 to position 390 of the ermB gene and does not cross-hybridize with any of the following genes: aataph, ant, aph, CMY1, CMY2, CTX1, CTX2, DHA, IMP, OXA, PER, SHV, TEM, VIM, ermA, ermC, mef, mecA, Spn pbp2b, Pae gyrA, Sau gyrA, Sau parC, Sau parE, vanA, and vanB;
an oligonucleotide set comprising an oligonucleotide of SEQ ID NOS: 88-92 or a complement thereof, wherein the oligonucleotide specifically hybridizes with a nucleotide region from position 40 to position 290 of the ermC gene and does not cross-hybridize with any of the following genes: aataph, ant, aph, CMY1, CMY2, CTX1, CTX2, DHA, IMP, OXA, PER, SHV, TEM, VIM, ermA, ermB, mef, mecA, Spn pbp2b, Pae gyrA, Sau gyrA, Sau parC, Sau parE, vanA, and vanB;
an oligonucleotide set comprising an oligonucleotide of SEQ ID NOS: 93-95 or a complement thereof, wherein the oligonucleotide specifically hybridizes with a nucleotide region from position 46 to position 288 of the mef gene and does not cross-hybridize with any of the following genes: aataph, ant, aph, CMY1, CMY2, CTX1, CTX2, DHA, IMP, OXA, PER, SHV, TEM, VIM, ermA, ermB, ermC, mecA, Spn pbp2b, Pae gyrA, Sau gyrA, Sau parC, Sau parE, vanA, and vanB;
an oligonucleotide set comprising an oligonucleotide of SEQ ID NOS: 96-101 or a complement thereof, wherein the oligonucleotide specifically hybridizes with a nucleotide region from position 2933 to position 3216 of the mecA gene and does not cross-hybridize with any of the following genes: aataph, ant, aph, CMY1. CMY2, CTX1, CTX2, DHA, IMP, OXA, PER, SHV, TEM, VIM, ermA, ermB, ermC, mef, Spn pbp2b, Pae gyrA, Sau gyrA, Sau parC, Sau parE, vanA, and vanB;
an oligonucleotide set comprising an oligonucleotide of SEQ ID NOS: 102-103 or a complement thereof, wherein the oligonucleotide specifically hybridizes with a nucleotide region from position 106 to position 442 of the vanA gene and does not cross-hybridize with any of the following genes: aataph, ant, aph, CMY1, CMY2, CTX1, CTX2, DHA, IMP, OXA, PER, SHV, TEM, VIM, ermA, ermB, ermC, mef, mecA, Spn pbp2b, Pae gyrA, Sau gyrA, Sau parC, Sau parE, and vanB;
an oligonucleotide set comprising an oligonucleotide of SEQ ID NOS: 104-105 or a complement thereof, wherein the oligonucleotide specifically hybridizes with a nucleotide region from position 847 to 1045 of the vanB gene and does not cross-hybridize with any of the following genes: aataph, ant, aph, CMY1, CMY2, CTX1, CTX2, DHA, IMP, OXA, PER, SHV, TEM, VIM, ermA, ermB, ermC, mef, mecA, Spn pbp2b, Pae gyrA, Sau gyrA, Sau parC, Sau parE, and vanA;
an oligonucleotide set comprising an oligonucleotide of SEQ ID NOS: 106, 108, 110, 112, 114, 116, 118, 120, and 122, or a complement thereof, wherein the oligonucleotide specifically hybridizes with a nucleotide region from position 399 to position 703 of the Pae wild-type gyrA gene and does not cross-hybridize with any of the following genes: aataph, ant, aph, CMY1, CMY2, CTX1, CTX2, DHA, IMP, OXA, PER, SHV, TEM, VIM, ermA, ermB, ermC, mef, mecA, Spn pbp2b, Sau gyrA, Sau parC, Sau parE, vanA, and vanB;
an oligonucleotide set comprising an oligonucleotide of SEQ ID NOS: 124, 126, 128, and 130, or a complement thereof, wherein the oligonucleotide specifically hybridizes with a nucleotide region from position 164 to position 317 of the Sau wild-type gyrA gene and does not cross-hybridize with any of the following genes: aataph, ant, aph, CMY1, CMY2, CTX1, CTX2, DHA, IMP, OXA, PER, SHV, TEM, VIM, ermA, ermB, ermC, mef, mecA, Spn pbp2b, Pae gyrA, Sau parC, Sau parE, vanA, and vanB;
an oligonucleotide set comprising an oligonucleotide consisting of at least 13 contiguous nucleotides present in a nucleotide sequence selected from the group consisting of SEQ ID NOS: 132, 134, and 136, or a complement thereof, wherein the oligonucleotide specifically hybridizes with a nucleotide region from position 38 to position 497 of the Sau wild-type parC gene and does not cross-hybridize with any of the following genes: aataph, ant, aph, CMY1, CMY2, CTX1, CTX2, DHA, IMP, OXA, PER, SHV, TEM, VIM, ermA, ermB, ermC, mef, mecA, Spn pbp2b, Pae gyrA, Sau gyrA, Sau parE, vanA, and vanB;
an oligonucleotide set comprising an oligonucleotide of SEQ ID NOS: 138 and 140 or a complement thereof, wherein the oligonucleotide specifically hybridizes with a nucleotide region from position 1166 to position 1501 of the Sau wild-type parE gene and does not cross-hybridize with any of the following genes: aataph, ant, aph, CMY1, CMY2, CTX1, CTX2, DHA, IMP, OXA, PER, SHV, TEM, VIM, ermA, ermB, ermC, mef, mecA, Spn pbp2b, Pae gyrA, Sau gyrA, Sau parC, vanA, and vanB; and
an oligonucleotide set comprising an oligonucleotide of SEQ ID NOS: 142, 144, 146, 148, and 150, or a complement thereof, wherein the oligonucleotide specifically hybridizes with a nucleotide region from position 294 to position 975 of the Spn wild-type pbp2b gene and does not cross-hybridize with any of the following genes: aataph, ant, aph, CMY1, CMY2, CTX1, CTX2, DHA, IMP, OXA, PER, SHV, TEM, VIM, ermA, ermB, ermC, mef, mecA, Pae gyrA, Sau gyrA, Sau parC, Sau parE, vanA, and vanB.

4. The microarray of claim 3, wherein the oligonucleotide probe set further comprises an oligonucleotide set consisting of:
an oligonucleotide set comprising oligonucleotides of SEQ ID NOS: 107, 109, 111, 113, 115, 117, 119, 121, and 123, or a complement thereof, wherein the oligonucleotide can specifically hybridize with a nucleotide region from position 399 to position 703 of the Pae mutant-type gyrA gene and does not cross-hybridize with any of the following genes: aataph, ant, aph, CMY1, CMY2, CTX1, CTX2, DHA, IMP, OXA, PER, SHV, TEM, VIM, ermA, ermB, ermC, mef, mecA, Spn pbp2b, Sau gyrA, Sau parC, Sau parE, vanA, and vanB;
an oligonucleotide set comprising oligonucleotides of SEQ ID NOS: 125, 127, 129, and 131, or a complement thereof, wherein the oligonucleotide can specifically hybridize with a nucleotide region from position 164 to position 317 of the Sau mutant-type gyrA gene and does not cross-hybridize with any of the following genes: aataph, ant, aph, CMY1, CMY2, CTX1, CTX2, DHA, IMP, OXA, PER, SHV, TEM, VIM, ermA, ermB, ermC, mef, mecA, Spn pbp2b, Pae gyrA, Sau parC, Sau parE, vanA, and vanB;
an oligonucleotide set comprising oligonucleotides of SEQ ID NOS: 133, 135, and 137, or a complement thereof, wherein the oligonucleotide can specifically hybridize with a nucleotide region from position 38 to position 497 of the Sau mutant-type parC gene and does not cross-hybridize with any of the following genes: aataph, ant, aph, CMY1, CMY2, CTX1, CTX2, DHA, IMP, OXA, PER, SHV, TEM, VIM, ermA, ermB, ermC, mef, mecA, Spn pbp2b, Pae gyrA, Sau gyrA, Sau parE, vanA, and vanB;
an oligonucleotide set comprising oligonucleotides of SEQ ID NOS: 139 and 141 or complementary thereof, wherein the oligonucleotide can specifically hybridize with a nucleotide region from position 1166 to position 1501 of the Sau mutant-type parE gene and does not cross-hybridize with any of the following genes: aataph, ant, aph, CMY1, CMY2, CTX1, CTX2, DHA, IMP, OXA, PER, SHV, TEM, VIM, ermA, ermB, ermC, mef, mecA, Spn pbp2b, Pae gyrA, Sau gyrA, Sau parC, vanA, and vanB; and
an oligonucleotide set comprising oligonucleotides of SEQ ID NOS: 143, 145, 147, 149, 151, 153, and 155, or a complement thereof, wherein the oligonucleotide can specifically hybridize with a nucleotide region from position 94 to position 975 of the Spn mutant-type pbp2b gene and does not cross-hybridize with any of the following genes: aataph, ant, aph, CMY1, CMY2, CTX1, CTX2, DHA, IMP, OXA, PER, SHV, TEM, VIM, ermA, ermB, ermC, mef, mecA, Pae gyrA, Sau gyrA, Sau parC, Sau parE, vanA, and vanB.

5. The microarray of claim 3, wherein the oligonucleotide probe set further comprises an oligonucleotide set consisting of:
an oligonucleotide set comprising oligonucleotides of SEQ ID NOS: 107, 109, 111, 113, 115, 117, 119, 121, and 123, or a complement thereof;
an oligonucleotide set comprising an oligonucleotides of SEQ ID NOS: 125, 127, 129, and 131, or a complement thereof;
an oligonucleotide set comprising oligonucleotides of SEQ ID NOS: 133, 135, and 137, or a complement thereof;
an oligonucleotide set comprising oligonucleotides of SEQ ID NOS: 139 and 141 or complementary thereof; and
an oligonucleotide set comprising oligonucleotides of SEQ ID NOS: 143, 145, 147, 149, 151, 153, and 155, or a complement thereof.


SEQUENCE LISTING (181 sequences)

SEQ ID NO: 1 - DNA, 22 bp, Artificial Sequence
  primer  forward
caagagcaataagggcatacca

SEQ ID NO: 2 - DNA, 22 bp, Artificial Sequence
  primer reverse
ctggcaatatctcgttttaaca

SEQ ID NO: 3 - DNA, 22 bp, Artificial Sequence
  primer  forward
tcaggtggatacttagagaaag

SEQ ID NO: 4 - DNA, 22 bp, Artificial Sequence
  primer  reverse
actatatatccgtgtcgttctg

SEQ ID NO: 5 - DNA, 22 bp, Artificial Sequence
  primer  forward
ggaccacctatgatgtggaacg

SEQ ID NO: 6 - DNA, 22 bp, Artificial Sequence
  primer reverse
gccgcttctcccaagatcaata

SEQ ID NO: 7 - DNA, 22 bp, Artificial Sequence
  primer forward
ataggatccgtgagcaagaccc

SEQ ID NO: 8 - DNA, 21 bp, Artificial Sequence
  primer reverse
cgcgcatcttctcggatgaat

SEQ ID NO: 9 - DNA, 21 bp, Artificial Sequence
  primer  forward
tacgctaactccagcattggt

SEQ ID NO: 10 - DNA, 21 bp, Artificial Sequence
  primer  reverse
cagcgggccatatcaataacg

SEQ ID NO: 11 - DNA, 21 bp, Artificial Sequence
  primer  forward
gtcaacggcacaatgacgctg

SEQ ID NO: 12 - DNA, 21 bp, Artificial Sequence
  primer  reverse
atcacccacagtccacgacgt

SEQ ID NO: 13 - DNA, 21 bp, Artificial Sequence
  primer  forward
ctgttgttaggaagtgtgccg

SEQ ID NO: 14 - DNA, 21 bp, Artificial Sequence
  primer reverse
cgtcagattccgcagagtttg

SEQ ID NO: 15 - DNA, 21 bp, Artificial Sequence
  primer forward
gtttggtgctctgaccgcaaa

SEQ ID NO: 16 - DNA, 23 bp, Artificial Sequence
  primer  reverse
acctggttgtctgttaccggatg

SEQ ID NO: 17 - DNA, 20 bp, Artificial Sequence
  primer forward
aaagacggtaaggttcaagc

SEQ ID NO: 18 - DNA, 20 bp, Artificial Sequence
  primer reverse
tcaagagtgatgcgtctcca

SEQ ID NO: 19 - DNA, 21 bp, Artificial Sequence
  primer forward
tttcgcaagaaataacccaaa

SEQ ID NO: 20 - DNA, 22 bp, Artificial Sequence
  primer  reverse
tttagaatggtgatcgcatttt

SEQ ID NO: 21 - DNA, 20 bp, Artificial Sequence
  primer  forward
attgcatttagctatgttgg

SEQ ID NO: 22 - DNA, 20 bp, Artificial Sequence
  primer  reverse
ataacaaatcacaggccacg

SEQ ID NO: 23 - DNA, 22 bp, Artificial Sequence
  primer  forward
gcgtaggcatgatagaaatgga

SEQ ID NO: 24 - DNA, 21 bp, Artificial Sequence
  primer  reverse
cagagttcgccgaccgtcatg

SEQ ID NO: 25 - DNA, 21 bp, Artificial Sequence
  primer  forward
gaccgaaggagctaaccgctt

SEQ ID NO: 26 - DNA, 22 bp, Artificial Sequence
  primer reverse
catagttgcctgactccccgtc

SEQ ID NO: 27 - DNA, 21 bp, Artificial Sequence
  primer  forward
accgacaacttagttgtgtac

SEQ ID NO: 28 - DNA, 21 bp, Artificial Sequence
  primer reverse
gtcggctgcaacttcatgtta

SEQ ID NO: 29 - DNA, 21 bp, Artificial Sequence
  primer  forward
agagctagtcaaaatgagtcg

SEQ ID NO: 30 - DNA, 21 bp, Artificial Sequence
  primer reverse
agaacacgatattcacggttt

SEQ ID NO: 31 - DNA, 21 bp, Artificial Sequence
  primer  forward
ttaacgacgaaactggctaaa

SEQ ID NO: 32 - DNA, 22 bp, Artificial Sequence
  primer reverse
aatatccaaggtacgcttgtag

SEQ ID NO: 33 - DNA, 22 bp, Artificial Sequence
  primer  forward
tttgtaatcagcacagttcatt

SEQ ID NO: 34 - DNA, 21 bp, Artificial Sequence
  primer reverse
gcagttacgaaattacacctc

SEQ ID NO: 35 - DNA, 22 bp, Artificial Sequence
  primer  forward
tatgggcagggcaagcagtatc

SEQ ID NO: 36 - DNA, 24 bp, Artificial Sequence
  primer reverse
tcrgcaccaatcattatcttcttc

SEQ ID NO: 37 - DNA, 22 bp, Artificial Sequence
  primer  forward
gcatgatttcttctgcaagttt

SEQ ID NO: 38 - DNA, 22 bp, Artificial Sequence
  primer reverse
ttcagttatttccccggacata

SEQ ID NO: 39 - DNA, 22 bp, Artificial Sequence
  primer  forward
tgaggaaggtagtaagggaaac

SEQ ID NO: 40 - DNA, 22 bp, Artificial Sequence
  primer reverse
ttgctacatactgagccaactg

SEQ ID NO: 41 - DNA, 20 bp, Artificial Sequence
  primer  forward
ccgccgtgtgctttatgcca

SEQ ID NO: 42 - DNA, 21 bp, Artificial Sequence
  primer reverse
ctcggtgccatcgtagttggg

SEQ ID NO: 43 - DNA, 22 bp, Artificial Sequence
  primer  forward
aacaaggtatgacaccggataa

SEQ ID NO: 44 - DNA, 22 bp, Artificial Sequence
  primer reverse
attgaaccaaagttaccttggc

SEQ ID NO: 45 - DNA, 21 bp, Artificial Sequence
  primer  forward
ttttaggtgatcgctttggaa

SEQ ID NO: 46 - DNA, 22 bp, Artificial Sequence
  primer reverse
gcagatatacctgtagaaccat

SEQ ID NO: 47 - DNA, 21 bp, Artificial Sequence
  primer  forward
cacgtaaagctcgtgaagatg

SEQ ID NO: 48 - DNA, 22 bp, Artificial Sequence
  primer reverse
catcagtatcagcatcagtcat

SEQ ID NO: 49 - DNA, 22 bp, Artificial Sequence
  primer  forward
tacgagccgttatacattggaa

SEQ ID NO: 50 - DNA, 22 bp, Artificial Sequence
  primer reverse
tattaataacccaaaaggcggg

SEQ ID NO: 51 - DNA, 21 bp, Artificial Sequence
  primer  forward
gatgatttgattgtcggcgaa

SEQ ID NO: 52 - DNA, 22 bp, Artificial Sequence
  primer reverse
cctgcaaaaaaagatcaacacg

SEQ ID NO: 53 - DNA, 24 bp, Artificial Sequence
  probe
taattcatgttctggcaaatcttc

SEQ ID NO: 54 - DNA, 24 bp, Artificial Sequence
  probe
tagtggttatgatagtgtggcata

SEQ ID NO: 55 - DNA, 24 bp, Artificial Sequence
  probe
taacaatcttcttttttgccctcg

SEQ ID NO: 56 - DNA, 24 bp, Artificial Sequence
  probe
gttatgaccatctgtgccagttcg

SEQ ID NO: 57 - DNA, 23 bp, Artificial Sequence
  probe
ctacgataagggcacaaatcgca

SEQ ID NO: 58 - DNA, 24 bp, Artificial Sequence
  probe
gaacttgtcttttcccacggcgac

SEQ ID NO: 59 - DNA, 24 bp, Artificial Sequence
  probe
gctttccttccagccatagcatca

SEQ ID NO: 60 - DNA, 22 bp, Artificial Sequence
  probe
caattccccgaggaggtggatt

SEQ ID NO: 61 - DNA, 21 bp, Artificial Sequence
  probe
gtggtcaagggagcgatgcag

SEQ ID NO: 62 - DNA, 24 bp, Artificial Sequence
  probe
accctcaggaatgagttacgaaga

SEQ ID NO: 63 - DNA, 24 bp, Artificial Sequence
  probe
tcttcgtaactcattcctgagggt

SEQ ID NO: 64 - DNA, 24 bp, Artificial Sequence
  probe
ggcggtgaaaccctcaggaatgag

SEQ ID NO: 65 - DNA, 21 bp, Artificial Sequence
  probe
ggacgatgtcactggctgagc

SEQ ID NO: 66 - DNA, 23 bp, Artificial Sequence
  probe
gacgtgcttttccgcaatcggat

SEQ ID NO: 67 - DNA, 23 bp, Artificial Sequence
  probe
gtattcagcgtaggttcagtgcg

SEQ ID NO: 68 - DNA, 23 bp, Artificial Sequence
  probe
atggcggtattcagcgtaggttc

SEQ ID NO: 69 - DNA, 23 bp, Artificial Sequence
  probe
attactgtgccggaaagtgcgca

SEQ ID NO: 70 - DNA, 23 bp, Artificial Sequence
  probe
atcattaacggtgtgaccaacga

SEQ ID NO: 71 - DNA, 20 bp, Artificial Sequence
  probe
tattattcggtggttgtttt

SEQ ID NO: 72 - DNA, 20 bp, Artificial Sequence
  probe
aactggttgttccaagtcac

SEQ ID NO: 73 - DNA, 20 bp, Artificial Sequence
  probe
aaatatggtaaggcaaaact

SEQ ID NO: 74 - DNA, 23 bp, Artificial Sequence
  probe
agccatgcttctgttaatccgtt

SEQ ID NO: 75 - DNA, 21 bp, Artificial Sequence
  probe
acgcaggaattgaatttgttc

SEQ ID NO: 76 - DNA, 20 bp, Artificial Sequence
  probe
gtaaacagggctaaggtttt

SEQ ID NO: 77 - DNA, 20 bp, Artificial Sequence
  probe
cagaatacctgggctccgat

SEQ ID NO: 78 - DNA, 21 bp, Artificial Sequence
  probe
gtgacgaacagctggagcgaa

SEQ ID NO: 79 - DNA, 21 bp, Artificial Sequence
  probe
gtggatgccggtgacgaacag

SEQ ID NO: 80 - DNA, 23 bp, Artificial Sequence
  probe
ctcgtcgtttggtatggcttcat

SEQ ID NO: 81 - DNA, 22 bp, Artificial Sequence
  probe
tggcttcattcagctccggttc

SEQ ID NO: 82 - DNA, 23 bp, Artificial Sequence
  probe
ctgagcgatttgtgtgcgctttt

SEQ ID NO: 83 - DNA, 22 bp, Artificial Sequence
  probe
ctcagtcgttgagtagcaggca

SEQ ID NO: 84 - DNA, 19 bp, Artificial Sequence
  probe
attaatggtggagatggat

SEQ ID NO: 85 - DNA, 23 bp, Artificial Sequence
  probe
tctgcaacgagctttgggtttac

SEQ ID NO: 86 - DNA, 21 bp, Artificial Sequence
  probe
gtggtttttgaaagccatgcg

SEQ ID NO: 87 - DNA, 21 bp, Artificial Sequence
  probe
tgcgtctgacatctatctgat

SEQ ID NO: 88 - DNA, 22 bp, Artificial Sequence
  probe
agagggttataatgaacgagaa

SEQ ID NO: 89 - DNA, 21 bp, Artificial Sequence
  probe
aaatacaaaacgctcattggc

SEQ ID NO: 90 - DNA, 24 bp, Artificial Sequence
  probe
aagagggttataatgaacgagaaa

SEQ ID NO: 91 - DNA, 21 bp, Artificial Sequence
  probe
tttgaaatcggctcaggaaaa

SEQ ID NO: 92 - DNA, 21 bp, Artificial Sequence
  probe
acaaaacgctcattggcatta

SEQ ID NO: 93 - DNA, 24 bp, Artificial Sequence
  probe
tgtctatggcttcattagtaggtt

SEQ ID NO: 94 - DNA, 24 bp, Artificial Sequence
  probe
ccatttgcaggatggcactagtga

SEQ ID NO: 95 - DNA, 24 bp, Artificial Sequence
  probe
tggcttcattagtaggttttttac

SEQ ID NO: 96 - DNA, 24 bp, Artificial Sequence
  probe
tgcttctgcaggatcttggtttgg

SEQ ID NO: 97 - DNA, 23 bp, Artificial Sequence
  probe
caagtgctaataattcacctgtt

SEQ ID NO: 98 - DNA, 21 bp, Artificial Sequence
  probe
gtatggcatgagtaacgaaga

SEQ ID NO: 99 - DNA, 22 bp, Artificial Sequence
  probe
aaatcagaatcaagaagtgctc

SEQ ID NO: 100 - DNA, 22 bp, Artificial Sequence
  probe
cagtacctgagccataatcatt

SEQ ID NO: 101 - DNA, 22 bp, Artificial Sequence
  probe
tttatgtatggcatgagtaacg

SEQ ID NO: 102 - DNA, 23 bp, Artificial Sequence
  probe
cattccgcgcaaggtttttcgca

SEQ ID NO: 103 - DNA, 22 bp, Artificial Sequence
  probe
cgttgacatacatcgttgcgaa

SEQ ID NO: 104 - DNA, 22 bp, Artificial Sequence
  probe
acggcaaagaaagtatatcggg

SEQ ID NO: 105 - DNA, 22 bp, Artificial Sequence
  probe
cctgatggatgcggaagatacc

SEQ ID NO: 106 - DNA, 20 bp, Artificial Sequence
  probe
aagaaatccgcccgwgtggt

SEQ ID NO: 107 - DNA, 20 bp, Artificial Sequence
  probe
aagaaatcctcccgwgtggt

SEQ ID NO: 108 - DNA, 22 bp, Artificial Sequence
  probe
aaatcckcycgtgtggtcggcg

SEQ ID NO: 109 - DNA, 22 bp, Artificial Sequence
  probe
aaatcckcycgagtggtcggcg

SEQ ID NO: 110 - DNA, 16 bp, Artificial Sequence
  probe
tcgccgtgcgggtggt

SEQ ID NO: 111 - DNA, 16 bp, Artificial Sequence
  probe
tcgccgtgtgggtggt

SEQ ID NO: 112 - DNA, 18 bp, Artificial Sequence
  probe
cggcgacaccscrgtcta

SEQ ID NO: 113 - DNA, 18 bp, Artificial Sequence
  probe
cggcgacatcscrgtcta

SEQ ID NO: 114 - DNA, 21 bp, Artificial Sequence
  probe
cscrgtctacgacaccatcgt

SEQ ID NO: 115 - DNA, 21 bp, Artificial Sequence
  probe
cscrgtctaccacaccatcgt

SEQ ID NO: 116 - DNA, 22 bp, Artificial Sequence
  probe
cacgatggtgtcgtagacygsg

SEQ ID NO: 117 - DNA, 22 bp, Artificial Sequence
  probe
cacgatggttgggtagacygsg

SEQ ID NO: 118 - DNA, 21 bp, Artificial Sequence
  probe
cscrgtctacgacaccatcgt

SEQ ID NO: 119 - DNA, 21 bp, Artificial Sequence
  probe
cscrgtctacaacaccatcgt

SEQ ID NO: 120 - DNA, 21 bp, Artificial Sequence
  probe
cscrgtctacgacaccatcgt

SEQ ID NO: 121 - DNA, 21 bp, Artificial Sequence
  probe
cscrgtctacaacaccatcgt

SEQ ID NO: 122 - DNA, 22 bp, Artificial Sequence
  probe
cscrgtctacgacaccatcgtc

SEQ ID NO: 123 - DNA, 22 bp, Artificial Sequence
  probe
cscrgtctacaacaccatcgtc

SEQ ID NO: 124 - DNA, 23 bp, Artificial Sequence
  probe
ctcatggtgactcayctatytat

SEQ ID NO: 125 - DNA, 23 bp, Artificial Sequence
  probe
ctcatggtgacttayctatytat

SEQ ID NO: 126 - DNA, 26 bp, Artificial Sequence
  probe wherein n represents inosine
catggtgactnatctatytatrnagc

SEQ ID NO: 127 - DNA, 26 bp, Artificial Sequence
  probe wherein n represents inosine
catggtgactnacctatytatrnagc

SEQ ID NO: 128 - DNA, 25 bp, Artificial Sequence
  probe wherein n represents inosine
tnayctatytatgaagcaatggtac

SEQ ID NO: 129 - DNA, 25 bp, Artificial Sequence
  probe wherein n represents inosine
tnayctatytataaagcaatggtac

SEQ ID NO: 130 - DNA, 24 bp, Artificial Sequence
  probe
cgtaccattgcttcataratagrt

SEQ ID NO: 131 - DNA, 24 bp, Artificial Sequence
  probe
cgtaccattgctccataratagrt

SEQ ID NO: 132 - DNA, 22 bp, Artificial Sequence
  probe
acayggagactcctcrgtgtac

SEQ ID NO: 133 - DNA, 22 bp, Artificial Sequence
  probe
acayggagacttctcrgtgtac

SEQ ID NO: 134 - DNA, 22 bp, Artificial Sequence
  probe
acayggagactcctcrgtgtac

SEQ ID NO: 135 - DNA, 22 bp, Artificial Sequence
  probe
acayggagactactcrgtgtac

SEQ ID NO: 136 - DNA, 21 bp, Artificial Sequence
  probe
accattgcttcgtacacygag

SEQ ID NO: 137 - DNA, 21 bp, Artificial Sequence
  probe
accattgctttgtacacygag

SEQ ID NO: 138 - DNA, 24 bp, Artificial Sequence
  probe
aaaaayacwgaaaaaaatgaattg

SEQ ID NO: 139 - DNA, 24 bp, Artificial Sequence
  probe
aaaaayacwgataaaaatgaattg

SEQ ID NO: 140 - DNA, 22 bp, Artificial Sequence
  probe
ccgattgtgtggataattgtat

SEQ ID NO: 141 - DNA, 22 bp, Artificial Sequence
  probe
ccgattgtgtagataattgtat

SEQ ID NO: 142 - DNA, 26 bp, Artificial Sequence
  probe wherein n represents inosine
tattcatchaatacctayatggtnca

SEQ ID NO: 143 - DNA, 26 bp, Artificial Sequence
  probe wherein n represents inosine
tattcatchaatgcttayatggtnca

SEQ ID NO: 144 - DNA, 25 bp, Artificial Sequence
  probe wherein n represents inosine
attcatcwaatacctayatggtnca

SEQ ID NO: 145 - DNA, 25 bp, Artificial Sequence
  probe wherein n represents inosine
attcatcwaatgcttayatggtnca

SEQ ID NO: 146 - DNA, 22 bp, Artificial Sequence
  probe wherein n represents inosine
cngctatggagaaaytkcgtnc

SEQ ID NO: 147 - DNA, 22 bp, Artificial Sequence
  probe wherein n represents inosine
cngctatgggaaaaytkcgtnc

SEQ ID NO: 148 - DNA, 16 bp, Artificial Sequence
  probe
gcttgggbactgcgac

SEQ ID NO: 149 - DNA, 16 bp, Artificial Sequence
  probe
gcttgggbgctgcgac

SEQ ID NO: 150 - DNA, 18 bp, Artificial Sequence
  probe
gcttgggbactgcgachg

SEQ ID NO: 151 - DNA, 18 bp, Artificial Sequence
  probe
gcttgggbgctgcgachg

SEQ ID NO: 152 - DNA, 16 bp, Artificial Sequence
  probe
gyttgggbactgcgac

SEQ ID NO: 153 - DNA, 16 bp, Artificial Sequence
  probe
gyttgggbtctgcgac

SEQ ID NO: 154 - DNA, 21 bp, Artificial Sequence
  probe wherein n represents inosine
tggyttgngbactgcgacngg

SEQ ID NO: 155 - DNA, 21 bp, Artificial Sequence
  probe wherein n represents inosine
tggyttgngbtctgcgacngg

SEQ ID NO: 156 - DNA, 1024 bp, Artificial Sequence
  nucleotide sequence of aataph gene
atgaatatagttgaaaatgaaatatgtataagaactttaatagatgatgattttcctttgatgttaaaatggttaactgatgaaagagtattagaattttatggtggtagagataaaaaatatacattagaatcattaaaaaaacattatacagagccttgggaagatgaagtttttagagtaattattgaatataacaatgttcctattggatatggacaaatatataaaatgtatgatgagttatatactgattatcattatccaaaaactgatgagatagtctatggtatggatcaatttataggagagccaaattattggagtaaaggaattggtacaagatatattaaattgatttttgaatttttgaaaaaagaaagaaatgctaatgcagttattttagaccctcataaaaataatccaagagcaataagggcataccaaaaatctggttttagaattattgaagatttgccagaacatgaattacacgagggcaaaaaagaagattgttatttaatggaatatagatatgatgatawtgccacaaatgttaaggcaatgaaatatttaattgagcattactttgataatttcaaagtagatagtattgaaataatcggtagtggttatgatagtgtggcatatttagttaataatgaatacatttttaaaacaaaatttagtactaataagaaaaaaggttatgcaaaagaaaaagcaatatataattttttaaatacaaatttagaaactaatgtaaaaattcctaatattgaatattcgtatattagtgatgaattatctatactaggttataaagaaattaaaggaacttttttaacaccagaaatttattctactatgtcagaagaagaacaaaatttgttaaaacgagatattgccagttttttaagacaaatgcacggtttagattatacagatattagtgaatgtactattgataataaacaaaatgtattagaagagtatatattgttgcgtgaaactatttataatg

SEQ ID NO: 157 - DNA, 771 bp, Artificial Sequence
  nucleotide sequence of ant gene
atgagaatagtgaatggaccaataataatgactagagaagaaagaatgaagattgttcatgaaattaaggaacgaatattggataaatatggggatgatgttaaggctattggtgtttatggctctcttggtcgtcagactgatgggccctattcggatattgagatgatgtgtgtcatgtcaacagargaagcagagttcagccatgaatggacaaccggtgagtggaaggtggaagtgaattttkatagcgaagagattctactagattatgcatctcaggtggaatcagattggcckcttacacatggtcaatttttctctattttgccgatttatgattcaggtggatacttagagaaagtgtatcaaactgctaaatcggtagaagcccaaamgttccacgatgcgatttgtgcccttatcgtagaagagctgtttgaatatgcaggcaaatggcgtaatattcgtgtgcaaggaccgacaacatttctaccatccttgactgtacaggtagcaatggcaggtgccatgttgattggtctgcatcatcgcatctgttatacgacgagcgcttcggtcttaactgaagcagttaagcaatcagatcttccttcaggttatgaccatctgtgccagttcgtaatgtctggtcaactttccgactctgagaaacttctggaatcgctagagaatttctggaatgggattcaggagtggacagaacgacacggatatatagtggatgtgtcaaaacgcataccattttga

SEQ ID NO: 158 - DNA, 1024 bp, Artificial Sequence
  nucleotide sequence of aph gene
ggaggggtcacgcgcaaatattaatatacctaaagatgaatttcaggattatgatattacatattttgtaagtgatatagaaccgtttatatctaatgatgactggcttaatcaatttgggaatataataatgatgcaaaagccggaggatatggaattattcccacctgaagaaaagggattttcctatcttatgctatttgatgattacaataaaattgatcttaccttattgcccttggaagagttagataattacctaaagggcgataaattaataaaggttctaattgataaagattgtagaattaaaagggacatagttccgactgatatagattatcatgtaagaaagccaagcgcaagggagtatgatgattgctgcaatgaattttggaatgtaacaccttatgttattaaaggattgtgccgtaaggaaattttatttgctattgatcattttaatcagattgttcgccatgagctgctgagaatgatatcatggaaggtcggcatcgaaacaggctttaaattaagtgtaggcaagaactataagtttattgaaaggtatatatccgaggatttgtgggagaaacttttgtccacctaccggatggattcctatgaaaacatatgggaagcattatttctatgccatcaattgttcagggcggtatccggtgaggtggcggaaaggcttcattatgcctatccggagtatgataggaatataacaaaatataccagggacatgtataaaaaatacactggtaaaaccggctgcctggatagcacatatgccgctgatatagaagagaggcgggaacagtgattacagaaatgaaagcagggcacctgaaagatatcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaat

SEQ ID NO: 159 - DNA, 1024 bp, Artificial Sequence
  nucleotide sequence of CMY1 gene
atgcaacaacgacaatccatcctgtggggggccgtggccaccctgatgtgggccggtctggcccatgcaggtgaggcttcaccggtcgatcccctgcgccccgtggtggatgccagcatccagccgctgctcaaggagcacaggatcccgggcatggcggtggccgtgctcaaggatggcaaggcccactayttcaattacggggtggccaaccgggagagcggggccrgcgtcagcgagcagaccctgttcgakataggatccgtgagcaagaccctgactgcgaccctgggggcctatgcggtggtcaagggagcgatgcagctggatgacaaggcgagccggcacgcgccctggctcaagggatccgyctttgacagcatcaccatgggggagcttgccacctacagcgccggaggcctgccactgcaattccccgaggaggtggattcatccgagaagatgcgcgcctactaccgccagtgggcccctgtctattcgccgggctcccatcgccagtactccaaccccagcatagggctgttcggccacctggcggcgagcagcctgaagcagccrtttgcccmsttgatggagcagaccctgctgcccgggctcggcatgcaccacacctatgtcaatgtgccgaagcaggccatggcgagttatgcctatggctattcgaaagaggacaagcccatccgkgtcaaccctggcatgctggcggacgaggcctayggcatcaagaccagctcggcggatctgctsssyttygtgaaggccaacatcggcggggttgatgacaaggcgttgcagcaggccatctccctgacccacmaagggcattactcggtaggcgggatgacccaggggctgggttgggagagttacgcctatcccgtcaccgagcagacattgctggcgggcaattcggccaaggtgakcctcgaagccaatccgacggcggckccccgggagtcggggagccaggtgctcttcaacaagaccggctcgaccaatggct

SEQ ID NO: 160 - DNA, 993 bp, Artificial Sequence
  nucleotide sequence of CMY2 gene, wherein n is unknown nucleotide
atgatgaaaaaatcgttatgctgcgctctgctgctgacagcctctttctccacatttgctgccgcaaaaacagaacaacagattgccgatatcgttaatcgcaccatcaccccgttgatgcaggagcaggctattccgggtatggccgttgccgttatctaccagggaaaaccctattatttcacctggggtaaagccgatatcgccaataaccacccagtcacgcagcaaacgctgtttgagctaggatcggttagtaagacgtttaacggcgtgttgggcggcgatgctatcgcccgcggcgaaattaagctcagcgatccggtcacgaaatactggccagaactgacaggcaaacagtggcagggtatccgcctgctgcacttagccacctatacggcaggcggcctaccgctgcagatccccgatgacgttagggataaagccgcattactgcatttttatcaaaactggcagccgcaatggactccgggcgctaagcgactttacgctaactccagcattggtctgtttggcgmgctggcggtgaaaccctcaggaatgagttacgaagaggcaatgaccagacgcgtcctgcaaccattaaaactggcgcatacctggattacggttccgcagaacgaacaaaaagattatgccwggggctatcgcgaagggaagcccgtacacgtttctccgggamaacttgacgccgaagcctatggcgtgaaatccagcgttattgatatggcccgctgggttcaggccaacatggatgccagccacgttcaggagaaaacgctccagcagggcattgcgcttgcgcagtctcgctactggcgtattggcgatatgtaccagggattaggctgggagatgctgaactggccgctgaaagctgattcgatcatcaacggcannnnnngcgacagcaaagtggcattggcagcgcttcccgccgttgaggtaaacccgcccgcccccgcagtgaaa

SEQ ID NO: 161 - DNA, 876 bp, Artificial Sequence
  nucleotide sequence of CTX1 gene
atggttaaaaaatcactgcgccagttcacgctgatggcgacggcaaccgtcacgctgttgttaggaagtgtgccgctgtatgcgcaaacggcggacgtacagcaaaaacttgccgaattagagcggcagtcgggaggcagactgggtgtggcattgattaacacagcagataattcgcaaatactttatcgtgctgatgagcgctttgcgatgtgcagcaccagtaaagtgatggccgcggccgcggtgctgaagaaaagtgaaagcgaaccgaatctgttaaatcagcgagttgagatcaaaaaatctgaccttgttaactataatccgattgcggaaaagcacgtcaatgggacgatgtcactggctgagcttagcgcggccgcgctacagtacagcgataacgtggcgatgaataagctgattgctcacgttggcggcccggctagcgtcaccgcgttcgcccgacagctgggagacgaaacgttccgtctcgaccgtaccgagcmgacgttaaacaccgccattccgggcgatccgcgtgataccacttcacctcgggcaatggcgcaaactctgcggaatctgacgctgggtaaagcattgggcgacagccaacgggcgcagctggtgacatggatgaaaggcaataccaccggtgcagcgagcattcaggctggactgcctgcttcctgggttgtgggggataaaaccggcagcggtgrctatggcaccaccaacgatatcgcggtgatctggccaaaagatcgtgcgccgctgattctggtcacttacttcacccagcctcaacctaaggcagaaagccgtcgcgatgtattagcgtcggcggctaaaatcgtcaccgacggtttgtaa

SEQ ID NO: 162 - DNA, 876 bp, Artificial Sequence
  nucleotide sequence of CTX2 gene
atggtgacaaagagagtgcaacggatgatgttcgcggcggcggcgtgcattccgctgctgctgggcagcgcgccgctttatgcgcagacgagtgcggtgcagcaaaagctggcggcgctggagaaaagcagcggagggcggctgggcgtcgcgctcatcgataccgcagataatacgcaggtgctttatcgcggtgatgaacgctttccaatgtgcagtaccagtaaagttatggcggccgcggcggtgcttaagcagagtgaaacgcaaaagcagctgcttaatcagcctgtcgagatcaagcctgccgatctggttaactacaatccgattgccgaaaaacacgtcaacggcacaatgacgctggcagaactgagcgcggccgcgttgcagtacagcgacaataccgccatgaacaaattgattgcccagctcggtggcccgggaggcgtgacggcttttgcccgcgcgatcggcgatgagacgtttcgtctggatcgcactgaacctacgctgaataccgccattcccggcgacccgagagacaccaccacgccgcgggcgatggcgcagacgttgcgtcagcttacgctgggtcatgcgctgggcgaaacccagcgggcgcagttggtgacgtggctcaaaggcaatacgaccggcgcagccagcattcgggccggcttaccgacgtcgtggactgtgggtgataagaccggcagcggcgactacggcaccaccaatgatattgcggtgatctggccgcagggtcgtgcgccgctggttctggtgacctattttacccagccgcaacagaacgcagagagccgccgcgatgtgctggcttcagcggcgagaatcatcgccgaagggctgtaa

SEQ ID NO: 163 - DNA, 1024 bp, Artificial Sequence
  nucleotide sequence of DHA gene
tgtaagtttttctttaggctcttgttataaataaccgtttgttctgtccggtgaatctgacgatacttgccgccgttactcacacacggaaggttaattctgatgaaaaaatcgttatctgcaacactgatttccgctctgctggcgttttccgccccggggttttctgccgctgataatgtcgcggcggtggtggacagcaccattaaaccgctgatggcacagcaggatattcccgggatggcggttgccgtctccgtaaagggtaagccctattatttcaattatggttttgccgatattcaggcaaaacagccggtcactgaaaatacactatttgagctcggatctgtaagtaaaactttcacaggtgtgctgggtgcggtttctgtggcgaaaaaagagatggcgctgaatgatccggcggcaaaataccagccggagctggctctgccgcagtggaaggggatcacattgctggatctggctacctataccgcaggcggactgccgttacaggtgccggatgcggtaaaaagccgtgcggatctgctgaatttctatcagcagtggcagccgtcccggaaaccgggcgatatgcgtctgtatgcaaacagcagtatcggcctgtttggtgctctgaccgcaaacgcggcggggatgccgtatgagcagttgctgactgcacggatcctggcaccgctggggttatctcacacctttattactgtgccggaaagtgcgcaaagccagtatgcgtacggttataaaaacaaaaaaccggtccgcgtgtcgccgggacagcttgatgcggaatcttacggcgtgaaatccgcctcaaaagatatgctgcgctgggcggaaatgaatatggagccgtcacgggccggtaatgcggatctggaaatggcaatgtatctcgcccagacccgctactataaaaccgccgcgattaaccaggggctgggctgggaaatgtatgactggccgcagcagaaagatatgatcattaacggtgtgacca

SEQ ID NO: 164 - DNA, 729 bp, Artificial Sequence
  nucleotide sequence of IMP gene
gtattctttatatttttgttttgyagcattgctaccgcagcagagyctttgccagatttaaaaattgaaaarcttgatgaaggcgtttatgttcatacttcgtttgaagaagttaacgggtggggcgttkttcctaaacatggtttggtkgttcttgtaratgctgargcttayctaattgacactccatttacggctaaagatactgaaaagttagtcacttggtttgtggarcgtggctataaaataaaaggcagyatttcctctcattttcatagygacagcacgggcggaatagagtggcttaattctcratcyatccccacgtatgcrtctgaattaacwaatgarctgcttaaaaaagacggtaaggttcaagcyamaaattcatttrgcggrgttaactattggctagttaaaaataaaattgaagttttttatccaggcccrggacacactccagataacstagtrgtttggytgcctgaaaggaaaatattattcggtggttgttttattaaaccgtacggtytaggyaatttgggtgacgcaaatwtagaagcttggccaaagtccgcyaaattattaawrtccaaatatggtaaggcaaaactggttgttccaagtcacagtgaagytggagacgcatcactcttgaaacttacattagagcaggcggttaaaggrttaaacgaaagtaaaaaaccatcaaaacyaagcaaytaawtt

SEQ ID NO: 165 - DNA, 998 bp, Artificial Sequence
  nucleotide sequence of OXA gene
gttgggcgaacccggagcctcattaattgttagccgttaaaattaagccctttaccaaaccaatacttattatgaaaaacacaatacatatcaacttcgctatttttttaataattgcaaatattatctacagcagcgccagtgcatcaacagatatctctactgttgcatctccattatttgaaggaactgaaggttgttttttactttacgatgyatccacaaacgctgaaattgctcaattcaataaagcaaagtgtgcaacgcaaatggcaccagattcaactttcaagatcgcattatcacttatggcatttgatgcggaaataatagatcagaaaaccatattcaaatgggataaaacccccaaaggaatggagatctggaacagcaatcatacaccaaagacgtggatgcaattttctgttgtttgggtttcgcaagaaataacccaaaaaattggattaaataaaatcaagaattatctcaaagattttgattatggaaatcaagacttctctggagataaagaaagaaacaacggattaacagaagcatggctcgaaagtagcttaaaaatttcaccagaagaacaaattcaattcctgcgtaaaattattaatcacaatctcccagttaaaaactcagccatagaaaacaccatagagaacatgtatctacaagatctggakaatagtacaaaactgtatgggaaaactggtgcaggattcacagcaaatagaaccttacaaaacggatggtttgaagggtttattataagcaaatcaggacataaatatgtttttgtgtccgcacttacaggaaacttggggtcgaatttaacatcaagcataaaagccaagaaaaatgcgatcaccattctaaacacactaaatttataaaaaatctaatggcaaaatcgcccaacccttcaatcaagtcgggacggccaaaagcaagcttttggctcccctcgctcggcgccccttatttcaaac

SEQ ID NO: 166 - DNA, 1024 bp, Artificial Sequence
  nucleotide sequence of PER gene
ttcaaaaatggttgaaaatgcggtaatctgattttgcttcattcgttttagccctctgggcgttctattttattcgcaaaatcaattagatcacgaatgaagcacctattcaaatcctaaagatcatacgtatgaaaaggacaatccgatgaatgtcattataaaagctgtagttactgcctcgacgctactgatggtatcttttagttcattcgaaacctcagcgcaatccccactgttaaaagagcaaattgaatccatagtcattggaaaaaaagccactgtaggcgttgcagtgtgggggcctgacgatctggaacctttactgattaatccttttgaaaaattcccaatgcaaagtgtatttaaattgcatttagctatgttggtactgcatcaggttgatcagggaaagttggatttaaatcagaccgttatcgtaaacagggctaaggttttacagaatacctgggctccgataatgaaagcgtatcagggagacgagtttagtgttccagtgcagcaactgctgcaatactcggtctcgcacagcgataacgtggcctgtgatttgttatttgaactggttggtggaccagctgctttgcatgactatatccagtctatgggtataaaggagaccgctgtggtcgcaaatgaagcgcagatgcacgccgatgatcaggtgcagtatcaaaactggacctcgatgaaaggtgctgcagagatcctgaaaaagtttgagcaaaaaacacagctgtctgaaacctcgcaggctttgttatggaagtggatggtcgaaaccaccacaggaccagagcggttaaaaggtttgttaccagctggtactgtggtcgcacataaaactggtacttcgggtatcaaagccggaaaaactgcggccactaatgatttaggtatcattctgttgcctgatggacggcccttgctggttgctgtttttgtgaaagactcagccgagtcaagccgaaccaatgaagctatcattgcgcaggttgctcag

SEQ ID NO: 167 - DNA, 861 bp, Artificial Sequence
  nucleotide sequence of SHV gene wherein n is unknown nucleotide.
atgcgttatwttcgcctgtgtattatctccctgttagccaccmtgccgctggcggtacacgccagcccgcagccgcttgagcaaattaaacwaagcgaaagccagctgtcgggcmgcgtaggcatgatagaaatggatctggccagcnnncgcacsctgaccgcctggcgcgccgatgracgctttcccatgatragcacctttaaagtagtgctctgcggcgcagtgctggcgcgggtggatgccggtgacgaacagctggagcgaaagatccactatcgccagcaggatctggtggactactcgccggtcagcgaaaaacaycttgccgacggcatgacggtcggcgaactctgygccgccgycattaccatgagcgataacagcgycgccaatctrctrytssssaccgtcggcggccccgyaggattgactgcctttttgcgccagatcgrcgacaacgtcacccgccttgaccgctgggaaacggaactgaatgaggcgcttcccggcgaygcccgcgacaccactaccccggccagcatggccgcgaccctgcgcaasstgctgaccagccagcgtctgagcgcccgttcgcaackgcagctgctgcagtggatggtggacgatcgrgtcgccggaccgttgatccgytccgtgctgycggcgggctggtttatcgccgataagaccggagctrscrarcggggtgcgcgcggsattgtcgccctgcttggcccgaataacaaagcagagcgsatygtggtgatttatctgcgggatacscyggcgagcatggccgagcgaaatcagcaaatcgccgggatcggcgcggcgctgatcgagcactggcaacgctaa

SEQ ID NO: 168 - DNA, 1024 bp, Artificial Sequence
  nucleotide sequence of TEM gene
atgagtattcaacatttycgtgtcgcccttattcccttttttgcggcattttgccttcctgtttttgctcacccagaaacgctggtgaaagtaraagatgctgaagatmagttgggtgcacgagtgggttacatcgarctggatctcaacagcggtaagatycttgagagttttcgccccgaagaacgttttccaatgntgagcacttttaaagttctgctatgtggygcggtattatcccgtgttgacgccgggcaagagcaactcggtcgccgcatacactattctcagaatgacttggttragtactcaccagtcacagaaaagcatcttacggatggcatgacagtaagagaattatgcartgctgccrtaaccatgrgtgataacactgckgccaacttacttctgacaacratcggaggaccgaaggagctaaccgcttttttgcrcaacatgggggatcatgtaacycgccttgatcrtygkgaaccggagctgaatgaagccataccaaacgacgagcgtgacaccacgaygcctgcagcaatggcaacaacgttgcgcaaactattaactggcgaactacttactctagcttcccrgcaacaattaatagactggatggaggcggataaagttgcaggaccacttctgcgctcggcccttccggctggctggtttattgctgataaatctggagcyrgtragcgtggrtctvgcggtatcattgcagcactggggccagatggtaagccctcccgtatcgtagttatctacacgacggggagtcaggcaactatggatgaacraratagacagatcgyygagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgataatgctctgccgc

SEQ ID NO: 169 - DNA, 913 bp, Artificial Sequence
  nucleotide sequence of VIM gene
gttatgccgcactcacccccatggagttttgatgttcaaacttttgagtaagttattggtctatttgaccgcgtctatcatggctattgcgagtccgctcgctttttccgtagattctagcggygagtatccgacagtcagcgaaattccggtcggggaggtccggctttaccagattgccgatggtgtttggtcgcatatcgcaacgcagtcgtttgatggcgcagtctacccgtccaatggtctcattgtccgtgatggtgatgagttgcttttgattgatacagcgtggggtgcgaaaaacacagcggcacttctcgcggagattgagaagcarattggacttcctgtaacgcgtgcagtctccacgcactttcatgacgaccgcgtcggcggcgttgatgtccttcgggcggctggggtggcaacgtacgcatcaccgtcgacacgccggctagccgaggtagaggggaacgagattcccacgcactctctagaaggactctcatcgagcggggacgcagtgcgcttyggtccagtagaactcttctatcctggtgctgcgcattcgaccgacaacttagttgtgtacgtcccgtctgcgagtgtgctctatggtggttgtgcgatttatgagttgtcacgcacgtctgcggggaacgtggccgatgccgatctggctgaatggcccacctccattgagcggattcaacaacactacccggaagcacagttcgtcattccggggcacggcctgccgggcggtctagacttgctcaagcacacaacgaatgttgtaaaagcgcacacaaatcgctcagtcgttgagtagcaggcagatgcggcataacatgaagttgcagccgaccatcactccgctgcgctccgttctggcggctgaacttcggcgtta

SEQ ID NO: 170 - DNA, 732 bp, Artificial Sequence
  nucleotide sequence of ermA gene
atgaaccagaaaaaccctaaagacacgcaaaattttattacttctaaaaagcatgtaaaagaaatattgaatcacacgaatatcagtaaacaagacaacgtaatagaaatcggatcaggaaaaggacattttaccaaagagctagtcaaaatgagtcgatcagttactgctatagaaattgatggaggcttatgtcaagtgactaaagaagcggtaaacccctctgagaatataaaagtgattcaaacggatattctaaaattttccttcccaaaacatataaactataagatatatggtaatattccttataacatcagtacggatattgtcaaaagaattacctttgaaagtcaggctaaatatagctatcttatcgttgagaagggatttgcgaaaagattgcaaaatctgcaacgagctttgggtttactattaatggtggagatggatataaaaatgctcaaaaaagtaccaccactatattttcatcctaagccaagtgtagactctgtattgattgttcttgarcgacatcaaccattgatttcaaagaaggactacaaaaagtatcgatcttttgtttataagtgggtaaaccgtgaatatcgtgttcttttcactaaaaaccaattccgacaggctttgaagcatgcaaatgtcactaatattaataaactatcgaaggaacaatttctttctattttcaatagttacaaattgtttcactaa

SEQ ID NO: 171 - DNA, 738 bp, Artificial Sequence
  nucleotide sequence of Spn ermB gene
atgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaacattcgtgtcactttaattcaccaagatattctacagtttcaattccctaacaaacagaggtataaaattgttgggaatattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataa

SEQ ID NO: 172 - DNA, 875 bp, Artificial Sequence
  nucleotide sequence of ermC gene
attttataaggaggaaaaaatatgggcatttttagtatttttgtaatcagcacagttcattatcaaccaaacaaaaaataagtggttataatgaatcgttaataagcaaaattcatataaccaaattaaagagggttataatgaacgagaaaaatataaaacacagtcaaaactttattacttcaaaacataatatagataaaataatgacaaatataagattaaatgaacatgataatatctttgaaatcggctcaggaaaaggscattttacccttgaattagtamagaggtgtaatttcgtaactgccattgaaatagaccataaattatgcaaaactacagaaaataaacttgttgatcacgataatttccaagttttaaacaaggatatattgcagtttaaatttcctaaaaaccaatcctataaaatatwyggtaatataccttataacataagtacrgatataatacgcaaaattgtttttgatagtatagctratgagatttatttaatcgtggaatacgrgtttgctaaaagattattaaatacaaaacgctcattggcattayttttaatggcagaagttgatatttctatattaagtatggttccaagagaatattttcatcctaaacctaaagtgaatagctcacttatcagattaaatagaaaaaaatcaagaatatcacacaaagataaacagaagtataattatttcgttatgaaatgggttracaaagaatacaagaaaatatttacaaaaaatcaatttaacaattccttaaaacatgcaggaattgacgatttaaacaatattagctttgaacaattcttatctcttttcaatagctataaattatttaataagtaa

SEQ ID NO: 173 - DNA, 1024 bp, Artificial Sequence
  nucleotide sequence of mef gene
aaattatggaaaaatacaacaattggaaacgaaaattttatgcaatatgggcagggcaagcagtatcattaatcactagtgccatcctgcaaatggcgattattttttaccttacagaaaaaacaggatctgcgatggtcttgtctatggcttcattagtaggttttttaccctatgcgattttgggacctgccattggtgtgctagtggatcgtcatgataggaagaagataatgattggtgccgatttaattatcgcagcagctggtgcagtgcttgctattgttgcattctgtatggagctacctgtctggatgattatgatagtattgtttatccgtagcattggaacagcttttcataccccagcactcaatgcggttacaccacttttagtaccagaagaacagctaacgaaatgcgcaggctatagtcagtctttgcagtctataagctatattgttagtccggcagttgcagcactcttatactccgtttgggatttaaatgctattattgccatcgacgtattgggtgctgtgattgcatctattacggtagcaattgtacgtatacctaagctgggtaatcaagtgcaaagtttagaaccaaatttcataagggagatgaaagaaggagttgtggttctgagacaaaacaaaggattgtttgccttattactcttaggaacactatatacttttgtttatatgccaatcaatgcactatttcctttaataagcatggaacactttaatggaacgcctgtgcatatttctattacggaaatttcctttgcatttgggatgctagcaggaggcttattattaggaagattagggggcttcgaaaagcatgtattactaataacaagttcattttttataatggggaccagtttagccgtttcgggaatacttcctccaaatggatttgtaatattcgtagtttgctgtgcaataatggggctttcggtgccattttatagcggtgtgcaaacagctctttttcaggagaaaattaa

SEQ ID NO: 174 - DNA, 1024 bp, Artificial Sequence
  nucleotide sequence of mecA gene
ctccatatcacaaaaattataacattattttgacataaatactacatttgtaatatactacaaatgtagtcttatataaggaggatattgatgaaaaagataaaaattgttccacttattttaatagttgtagttgtcgggtttggtatatatttttatgcttcmaaagataaagaaattaataatactattgatgcaattgaagataaaaatttcaaacaagtttataaagatagcagttatatttctaaaagcgataatggtgaagtagaaatgactgaacgtccgataaaaatatataatagtttaggcgttaaagatataaacattcaggatcgtaaaataaaaaaagtatctaaaaataaaaaacgagtagatgctcaatataaaattaaaacaaactacggtaacattgatcgcaacgttcaatttaattttgttaaagaagatggtatgtggaagttagattgggatcatagcgtcattattccaggaatgcagaaagaccaaagcatacatattgaaaawttaaaatcagaacgtggtaaaattttagaccgaaacaatgtggaattggccaatacaggaacagcatatgagattaggcatcgttccaaagaatgtatctaaaaaagattataaagcaatcgctaaagaactaagtatttctgaagactatatcaaacaacaaatggatcaaaaktgggtacaagatgataccttcgttccactttaaaaccgttaaaaaaatggatgaatatttaagkgatttcgcaaaaaaatttcatcttacaactaatgaaacagaaagtcgtaactatcctctagraaaagcgacttcacatctattaggttatgttggtcccattaactctgaagaattaaaacaaaaagaatataaaggctataaagatgatgcagttattggtaaaaagggactcgaaaaactttacgataaaaagctccaacatgaagatggctatcgtgtcacaatcgttgacgataatagcaatacaatcgcaca

SEQ ID NO: 175 - DNA, 1024 bp, Artificial Sequence
  nucleotide sequence of pbp2b gene
atggctgttattgcctctatttcaaaggagatgcctggcattagtatttctacttcttgggatagaaaggttttrgaaacytcyctttcttctatagtwggkagtgtatccagtgaaaaagctggtctcccagcggaagaagyrgawrcctatcttaaaaaaggytattctctaaatgaycgtgtwggaacctcctatttggaaaagcaatatgaagagaccttacaaggraaacgctcggtaaaagaaatccatctggataaatatggcaayatggaaagygtggatacaattgaggaaggtagtaagggaaacaatatcaaactgaccattgatttggctttcaagatagygtggatgctttrytgaaaagttatttcaattcygagctagraaatggkggagccaagtattctgarggtgtytatgcagtygcyytkaayccmaaaacaggtgckgttttgtctatgtcaggrmtyaaacatgacytgaamacgggagagttrackcckgattccttgggaacggtaaccaatgtctttgtyccaggktcrgtwgtyaaggcbgckaccatcagctcwggytgggaaaatggwgtyttrtcaggaaaccaraccttracagaycagycyattgtyttycaaggttcakctccmatywattcttggtatamwydggcwtayggwtcwttycctatyacagcdgtssaagcyytggagtattcatcwaatrsytayatggtycaaacmgcyytwggwmtyatgggscaracytaycaaccmaatatgtttgtyggmaccagcaatytrgarwcwgctatggrraaaytkcgtkcracctttggcgaatatggcttgggbdctgcgachggrattgacctaccagatgaatctactggatttgttcccaaagagtatagctttgctaattacatyacyaatgcctttgggcagtttgataactatacgccsatgcagttggctcagtatgtagcaactattgcaaatratggtgttcgtgtggc

SEQ ID NO: 176 - DNA, 1024 bp, Artificial Sequence
  nucleotide sequence of Pae gyrA gene wherein n is unknown nucleotide.
nnnnnnnvvnvnnnnnnnnntgcattgaacgaggcgactggaggtcgtccccgccagggcctttgccttgggctggggcgtgggctgtggtaagctccgacggttattcgagcgccccgcggaggggcctcgagagtgcgcgaatccttgactcaagtcgttgatttgtagtgagttggcgctgctcgggcatgcgccgacctacttcgtttgcctcaggatcgaggcggcgaagttccaccagaaaaaggaaccaggcttctcatgggcgaactggccaaagaaattctcccggtcaatatcgaagacgagctgaaacagtcctatctcgactacgcgatgagcgtgatcgtcgggcgggccctgccggatgcacgtgacggcctgaagccggtgcaccgccgtgtgctttatgccatgagcgagctgggcaacgactggaacaagccctacaagaaatcckcycgwgtggtcggcgacgtgatcggtaagtaccacccrcacggcgacaycscrgtctacvvmaccatcgtgcgyatggcgcagccgttctcgctgcgctacatgctggtagacggccwgggcaacttcggttcggtggacggcgacaacgccgcagccatgcgatacaccgaagtgcgcatggccaagctggcccacgaactgctggcggacctggaaaaggaaaccgtcgactgggtgcccaactacgatggcaccgagcagatcccggcggtcatgccgaccaagattcccaacctgctggtcaacggttccagcggtatcgccgtgggcatggcgaccaacatcccgccgcacaacctcggcgaagtgatcgacggctgcctggcgctgatggacaaccccgacctgaccgtcgatgagctgatgcagtacatccccggtccggacttccccaccgccggcatcatcaacggccgcgccgggatcatcgaggcctaccgcaccggtcgcgggcgcatctacatccgtgcccgcgccgtcgtcgaggagatggagaagg

SEQ ID NO: 177 - DNA, 1024 bp, Artificial Sequence
  nucleotide sequence of Sau gyrA gene
atggctgaattacctcaatcaagaataaatgaacgaaatattaccagtgaaatgcgtgaatcatttttagattatgcgatgagygttatcgttgctcgtgcattgccagatgttcgtgacggtttaaaaccagtacatcgtcgtatactatatggattaaatgaacaaggtatgacaccggataaatcatataaaaaatcagcacgtatcgttggtgacgtaatgggtaaatatcaccctcatggtgactyayctatytatrragcaatggtacgtatggctcaagatttcagttatcgttatccgctkgttgatggccaaggtaactttggttcaatggatggagatggcgcagcagcaatgcgttatactgaagcrcgtatgactaaaatcacacttgaactgttacgtgatattaataaagatacaatagattttatcgataactatgatggtaatgaaagagagccgtcagtcttacctgctcgattccctaayttrttagccaatggwgcatcaggtatmgcggtaggtatggcaacgaatattccaccacataacttaacagaattratcaatggtgtacttagcttaagtaagaaycctgatatttcaattgctgagttaatggargatattgaaggtcctgatttcccwactgctggacttattttaggtaagagtggtattagacgygcatatgaaacaggtcgtggttcaattcaaatgcgttctcgtgcagttattgaagaacgtggaggcsgacgtcaacgtattgttgtcactgaaattcctttccaagtgaataaggctcgtatgattgaaaaaattgcagarctygttcgtgacaagaaaattgacggtatyactgatttacgtgatgaaacaagtttacgtactggtgtgcgtgtcgttattgatgtgcgtaaggatgcmaatgctagtgtcattttaaataacttatacaaacaaacrccwcttcaaacatcatttggtgtgaatatgattgcwctwgtraatggta

SEQ ID NO: 178 - DNA, 1024 bp, Artificial Sequence
  nucleotide sequence of Sau ParC
gtgagtgaaataattcaagatttatcacttgaagatgttttaggtgatcgctttggaagatatagtaaatatattattcaagagcgtgcattgccagatgttcgtgatggtttaaaaccmgtacaacgtcgtattttataygcaatgtattcaagtggtaatacacacgataaaaatttccgtaaaagtgcgaaaacagtcggtgatgttattggtcaatatcatccacatggagacthctcagtgtacrragcaatggtccgtttaagtcaagactggaagttacgacatgtcttaatagaaatgcatggtaataatggtagtatcgataatgatccrccagcggcaatgcgttacactgaagctaagttaagcytactagctgaagagttattacgtgatattaataaagagacagtttcyttcattycaaactatgatgatacgacrctcgaaccaatggtattgccatcaagatttcctaacttactagtgaatggttctacaggtatatctgcaggttacgcgacagatataccaccacataatttagctgaagtgattcaagcaacacttaaatatattgataatccrgatattacagtcaatcaattaatgaaatatattaaaggtcctgattttccaactggyggtattattcaaggtattgatggtattaaaaaagcttatgaatcaggtaaaggtagaattatagttcgttctaaagttgaagaagaaactttacgcaatggacgtaaacagttaattattactgaaattccatatgaagtgaacaaargtagcttagtaaaacgtatcgatgaattacgtgctgacaaaaaagtcgatggtatcgttgaagtacgtgatgaaactgatagaactggtttacgaatagcaattgaattgaaaaaagatgtgaacagtgaatcaatcaaaaattatctttataaaaactctgatttacagatttcatataatttcaacatggtcgctattagtgatggtcgtccaaaattgatgg

SEQ ID NO: 179 - DNA, 1024 bp, Artificial Sequence
  nucleotide sequence of Sau ParE gene
atgaataaacaaaataattattcagatgattcaatacaggttttagaggggttagaagcagttcgtaaaagacctggtatgtatattggatcaactgataaacggggattacatcatctagtatatgaaattgtcgataactccgtcgatgaagtattgaatggttacggtaacgaaatagatgtaacaattaataaagatggtagtatttctatagaagataatggacgtggtatgccaacaggtatacataaatcaggtaaaccgacagtcgaagttatctttactgttttacatgcaggaggtaaatttggacaaggyggctataaaacttcaggtggtcttcacggygttggtgcttcagtkgtaaatgcattgagtgaatggcttgaagttgaaatccatcgagatggtartatatatcatcaaagttttaaaaacggtggttcgccatcttcwggtttagtgaaaaaaggtaaaactaagaaaacaggtaccaaagtaacatttaaacctgatgacacaatttttaaagcatctacatcatttaattttgatgttttaagygaacgactacaagagtctgcgttcttattgaaaaatttaaaaataacgcttaatgatttacgcagtggtaaagagcgtcaagagcattaccattatgaagaaggaatcaaagagtttgttagttatgtcaatgaaggaaaagaagttttgcatgacgtggctacattttcaggtgaagcaaatggtatagaggtagacgtagctttccaatataatgatcaatattcagaaagtattttaagttttgtaaataatgtacgtactaaagatggtggtacacatgaagttggttttaaaacagcaatgacacgygtatttaatgattatgcacgtcgtattaatgaacttaaaacaaaagataaaaacttagatggtaatgatattcgtgaaggtttaacagctgttgtgtctgttcgtattccagaagaattattrcaatttgaaggacaaacgaaat

SEQ ID NO: 180 - DNA, 1024 bp, Artificial Sequence
  nucleotide sequence of VanA gene
atgaatagaataaaagttgcaatactgtttgggggttgctcagaggagcatgacgtatcggtaaaatctgcaatagagatagccgctaacattaataaagaaaaatacgagccgttatacattggaattacgaaatctggtgtatggaaaatgtgcgaaaaaccttgcgcggaatgggaaaacgacaattgctattcagctgtactctcgccggataaaaaaatgcacggattacttgttaaaaagaaccatgaatatgaaatcaaccatgttgatgtagcattttcagctttgcatggcaagtcaggtgaagatggatccatacaaggtctgtttgaattgtccggtatcccttttgtaggctgcgatattcaaagctcagcaatttgtatggacaaatcgttgacatacatcgttgcgaaaaatgctgggatagctactcccgccttttgggttattaataaagatgataggccggtggcagctacgtttacctatcctgtttttgttaagccggcgcgttcaggctcatccttcggtgtgaaaaaagtcaatagcgcggacgaattggactacgcaattgaatcggcaagacaatatgacagcaaaatcttaattgagcaggctgtttcgggctgtgaggtcggttgtgcggtattgggaaacagtgccgcgttagttgttggcgaggtggaccaaatcaggctgcagtacggaatctttcgtattcatcaggaagtcgagccggaaaaaggctctgaaaacgcagttataaccgttcccgcagacctttcagcagaggagcgaggacggatacaggaaacggcaaaaaaaatatataaagcgctcggctgtagaggtctagcccgtgtggatatgtttttacaagataacggccgcattgtactgaacgaagtcaatactctgcccggtttcacgtcatacagtcgttatccccgtatgatggccgctgcaggtattgcacttcccgaactgattgaccgcttgatcgtattagcgttaa

SEQ ID NO: 181 - DNA, 1024 bp, Artificial Sequence
  nucleotide sequence of VanB gene wherein n is unkown nucleotide
tcagtttgtttataccgattgctcgcagaaagtgcttgaccatccctttttgtcgcagcttttaaggatgccgaatgtgatcatcacaccccatacggcgtactacactgagcgtgtgctgcrrgatacyacagaaaanncaatcaggaattgtctyaaytttgaaaggagtttacagcatgaataraataaaagtcgcaatyatcttcggcggttgctcggaggaacatgatgtgtcggtaaaatccgcaatagaaattgctgcgaacattratackgaaaaattcgatccgcactacatcggaattacaaaaarsggygtatggaagctatgcaagaagccatgtacggaatgggaagccgayagtctccccgccataytctccccggataggaaaacgcatggkctgcttgtcatgaaagaaagmgaatacgaaacwcggcgtattgaygtggctttcccrgttttgcatggcaaatgcggggaggayggntgcgatmcagggdytrtttgwattgtctggyatcccctatgtrggctgygatattcaaagctccgcagyttgcrtggacaaatcactggcctacattcttacaaaaaatgcgggcatcgccgtycccgaatttcaaatkattgawaaaggtgacaarccggagrcgrgkrcgcttacctaccctgtctttgtgaagccggcacggtcaggttcgtcctttggcktaaccaaagtaaacrgtacggaagaactwaacgctgcgatagaagcrgcaggacaatatgatggaaaaatcttaattgagcaagcgatttcgggctgtgaggtcggstgygcggtyatgggraacgaggatgatttgattgtcggcgaagtggatcaaatccggytgagccayggtatcttccgcatccatcaggaaaacgagccggaaaaaggmtcagaraatgcgatgattaymgttcchgcagacatyccrgtcgrggaacgaaawcgggtgcargaaacggcaaagaaagtatatcgggtgc